(19) United States
(12) Patent Application Publication
(10) Pub. No.: US 2025/0302817 A1
(43) Pub. Date: Oct. 2, 2025

(54) METHODS FOR TREATING PTEN-MUTANT TUMORS

(71) Applicant: Icahn School of Medicine at Mount Sinai, New York, NY (US)

(72) Inventors: Ramon E. Parsons, Manhasset, NY (US); Deepti Mathur, Colombus, OH (US); Ilias Stratikopoulos, New York, NY (US)

(21) Appl. No.: 18/943,681

(22) Filed: Nov. 11, 2024

Related U.S. Application Data
(63) Continuation of application No. 18/434,463, filed on Feb. 6, 2024, now abandoned.
Application No. 18/434,463 is a continuation of application No. 18/329,440, filed on Jun. 5, 2023, now abandoned.
Application No. 18/329,440 is a continuation of application No. 17/476,353, filed on Sep. 15, 2021, now abandoned.
Application No. 17/476,353 is a continuation of application No. 16/327,185, filed on Feb. 21, 2019, now abandoned.
(60) Provisional application No. 62/378,404, filed on Aug. 23, 2016.

(51) Int. Cl. A61K 31/47; A61K 31/277; A61K 31/42; A61K 45/06; A61P 35/00; C12Q 1/68; C12Q 1/6886; G01N 33/574

(52) U.S. Cl. CPC A61K 31/47; A61K 31/277; A61K 31/42; A61K 45/06; A61P 35/00; C12Q 1/68; C12Q 1/6886; G01N 33/574; C12Q 2600/106; C12Q 2600/156; G01N 2800/52; G01N 2800/7028

(57) ABSTRACT

Methods for assessing the efficacy of dihydroorotate dehydrogenase inhibitors in the treatment of cancer and methods of using such inhibitors to treat PTEN-mutant cancer are provided.

### CROSS-REFERENCE TO RELATED APPLICATIONS

This application is a continuation of pending U.S. application Ser. No. 18/434,463, filed Feb. 6, 2024, which is a continuation of U.S. application Ser. No. 18/329,440, filed Jun. 5, 2023, now abandoned, which is a continuation of U.S. application Ser. No. 17/476,353, filed Sep. 15, 2021, now abandoned, which is a continuation of U.S. application Ser. No. 16/327,185, filed Feb. 21, 2019, now abandoned, which is a U.S. National Stage application of International Application No. PCT/US2017/045085, filed Aug. 2, 2017, which claims priority to U.S. Provisional Application No. 62/378,404, filed Aug. 23, 2016. The contents of all of the prior applications are incorporated by reference herein in their entirety.

### GOVERNMENT GRANT CLAUSE

This invention was made with government support under grant nos. CA097403, CA082783, and CA155117 awarded by the National Institutes of Health. The government has certain rights in the invention.

### TECHNICAL FIELD

This disclosure relates to compositions and methods for administering one or more dihydroorotate dehydrogenase (DHODH) inhibitors to a subject for the treatment of phosphatase and tensin homolog (PTEN)-mutant tumors, and to methods of predicting the efficacy of a DHODH inhibitor in treating cancers.

### BACKGROUND OF THE INVENTION

The Warburg effect is a classic metabolic alteration of cancer cells, changing the way cells take up and process glucose to drive tumor growth. Studies have found that glutamine is also vital for growth, fueling the synthesis of tricarboxylic acid cycle intermediates, phospholipid and nucleotide synthesis, and NADPH. Oncogenic signaling pathways have been shown to play a major role in reprogramming glucose and glutamine metabolism, thus connecting genetic mutations with metabolic alterations. PTEN (phosphatase and tensin homolog deleted on chromosome 10) is one of the most commonly mutated tumor suppressors and is a fulcrum of multiple cellular functions. PTEN's canonical role is as a lipid phosphatase for phosphatidylinositol-3,4,5-trisphosphate, central to the phosphoinositide-3 kinase (PI3K) pathway, limiting AKT, mTOR, and RAC signaling. Inactivation of PTEN enhances glucose metabolism and diminishes DNA repair and DNA damage checkpoint pathways. Furthermore, deficient homologous recombination in PTEN-mutant cells leads to sensitivity to gamma-irradiation and PARP inhibitors. The role of PTEN in metabolism, however, has not been completely examined.

Many different types of cancer (e.g., breast cancer (e.g., triple-negative breast cancer), bladder cancer, colon/colorectal cancer, uterine cancer, ovarian cancer, glioblastoma multiforme, prostate cancer, pancreatic cancer, melanoma, renal cell carcinoma, lymphoma, leukemia, oropharyngeal cancer, etc.) can comprise mutations that inactivate the PTEN tumor suppressor. Alteration of PTEN can either be inherited (germline) or somatic within a cancer. The frequency of inactivation of PTEN varies among different tumor types. PTEN is most frequently inactivated in triple-negative breast cancer, uterine cancer, and advanced cancer of the prostate and brain.

Triple-negative breast cancer (TNBC) subtype represents about 15% of breast cancers and is characterized by the lack of expression of estrogen receptor (ER), progesterone receptor (PR) and HER-2 non-amplification. Women with TNBC tend to be younger, African-American, and BRCA-1 germline carriers. The hallmark of this subtype is early metastatic recurrences with a peak frequency 1-2 years. Prognosis for metastatic TNBC is especially poor, with median survival of about 1 year relative to about 2-4 years with other subtypes of metastatic breast cancer. TNBCs are not uniform, but rather comprise a family of distinct cancers that can be characterized by unique expression profiling. There is no standard or targeted chemotherapy for metastatic TNBC. Both TNBC and BRCA-1 associated breast cancers are sensitive to DNA cross-linking agents such as platinum compounds and more recently, the androgen receptor inhibitors and checkpoint inhibitors have shown some activity in treating TNBC. There remains a critical need to identify additional targets and biomarkers that are predictive of response in subsets of TNBC.

### SUMMARY

The present disclosure provides methods of predicting the efficacy of a DHODH inhibitor in inducing DNA damage in PTEN-mutant cancer cells, and methods for the treatment of PTEN-mutant cancer.

The disclosure provides a method for the treatment of a subject (e.g., a human subject) having a phosphatase and tensin homolog (PTEN)-mutant cancer, the method including administering to a subject with a PTEN-mutant cancer at least one dihydroorotate dehydrogenase (DHODH) inhibitor. In one aspect, the disclosure provides a method for the prevention of a phosphatase and tensin homolog (PTEN)-mutant cancer in a subject (e.g., a human subject) at risk thereof, the method including administering to a subject at risk of developing a PTEN-mutant cancer at least one dihydroorotate dehydrogenase (DHODH) inhibitor. The PTEN-mutant cancer can be, e.g., breast cancer (e.g., triple-negative breast cancer), a glioblastoma, prostate cancer, uterine cancer, ovarian cancer, pancreatic cancer, melanoma, thyroid cancer, renal cell carcinoma, bladder cancer, colorectal cancer, lymphoma, leukemia, and/or oropharyngeal cancer. The PTEN-mutant cancer can be a relapsed cancer. The PTEN-mutant cancer can have been refractory to one or more previous treatments. The PTEN-mutant cancer can be partially deficient for PTEN or active PTEN relative to a wild-type tissue of the same species and tissue type. The PTEN-mutant cancer can lack detectable PTEN or active PTEN. PTEN inactivation can occur through any combination of inherited or acquired mutations or deletions.

The disclosure also features a method for predicting the efficacy of a DHODH inhibitor in inducing DNA damage in a cancer, the method including testing a cell of the cancer for the presence of wild-type or mutant PTEN, and predicting that a DHODH inhibitor would likely induce DNA damage in the cancer if the cell is partially deficient for PTEN or active PTEN relative to a wild-type cell of the same species and tissue type, or if the cell does not contain detectable PTEN or active PTEN. The method can include, if the cancer cell is found to be partially deficient for PTEN or active PTEN relative to a wild-type cell of the same species and tissue type, or if the cancer cell does not express detectable PTEN or active PTEN, administering to a subject with the cancer at least one DHODH inhibitor.

The disclosure also features a method for a method of adjuvant therapy comprising administering to a human subject with phosphatase and tensin homolog (PTEN)-mutant cancer, following primary therapy an effective amount of one or more dihydroorotate dehydrogenase (DHODH) inhibitors. Adjuvant therapy, in the broadest sense, is treatment given in addition to the primary therapy (e.g., primary chemotherapy or definitive surgery), to kill any cancer cells that may have spread, even if the spread cannot be detected by radiologic or laboratory tests. In some embodiments, the one or more dihydroorotate dehydrogenase (DHODH) inhibitors is administered with one or more chemotherapeutic agents.

Also provided by the disclosure is a method for predicting the efficacy of a DHODH inhibitor in treating a cancer, the method including testing a cell of the cancer for the presence of wild-type or mutant PTEN, and predicting that a DHODH inhibitor would likely induce DNA damage in the cancer and thereby treat the cancer if the cell is partially deficient for PTEN or active PTEN relative to a wild-type cell of the same species and tissue type, or if the cell does not comprise detectable PTEN or active PTEN. The method can include, if the cancer cell is found to be partially deficient for PTEN or active PTEN relative to a wild-type cell of the same species and tissue type, or if the cancer cell does not express detectable PTEN or active PTEN, administering to a subject with the cancer at least one DHODH inhibitor.

In any of the above-described methods, at least one DHODH inhibitor can be, e.g., one or more of brequinar, leflunomide, redoxal, S-2678, and/or teriflunomide (also known as A771726). At least one DHODH inhibitor can be, e.g., administered orally, or via any other route known in the art (e.g., parenterally, intradermally, subcutaneously, topically, or rectally).

Any of the above-described methods can further include treating the subject with one or more additional therapeutic regimens. The one or more additional therapeutic regimens can be, e.g., one or more of surgery, chemotherapy, radiation therapy, hormone therapy, and/or immunotherapy.

As used herein, the terms “about” and “approximately” are defined as being within plus or minus 10% of a given value or state, preferably within plus or minus 5% of said value or state.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a graph comparing the growth of Pten wild-type (WT) and KO MEFs (one-way ANOVA, *p<0.0001, n=3). FIG. 1B is a series of representative confocal microscopy photographs showing MEFs labeled with EdU. FIG. 1C is a graph showing a quantitative depiction of the photographs in FIG. 1B (Student's t-test, *p<0.05, n=6). FIG. 1D is a graph showing a quantitative depiction of the mean fluorescence intensity of MEFs labeled with EdU; cells were analyzed by flow cytometry (Student's t-test, *p<0.01, n=3). FIG. 1E is a series of representative photographs showing Pten WT and KO MEFs in media containing full glutamine (6 mM) or no added glutamine (one-way ANOVA, *p<0.0001, n=3). FIG. 1F is a series of representative photographs showing MEFs treated with 12.5 nM CB-839 or control (one-way ANOVA, *p<0.0001, n=3). FIG. 1G is a graph comparing the relative metabolite concentrations of DNA nucleotide precursors in Pten WT and KO MEFs (dGMP was unable to be measured so dGTP was used instead) (Student's t-test, *p<0.05, n=3). FIG. 1H is a graph comparing the relative metabolite levels of glutamine-labeled de novo pyrimidine synthesis intermediates in Pten WT and KO MEFs (Student's t-test, *p<0.05, n=3). Data were also analyzed with IMPaLA: ¹³C glutamine-derived pyrimidine metabolism enrichment in PTEN^{-/-} MEFs q-value=3.92×10⁻⁰⁹. Data shown as mean+SD.

FIG. 2A is a graph showing the GI50s of Pten WT and KO cells treated with dose titrations of leflunomide, A771726 (teriflunomide), or brequinar (Student's t-test, *<0.05, n=3). FIGS. 2B and 2C are graphs showing the GI50s of various cells treated with dose titrations of leflunomide (Student's t-test, *p-values on figures, n=3). FIGS. 2D and 2E are graphs depicting the accumulation of cell death in 6 h intervals of cells treated with 100 uM leflunomide and DRAQ7 (one-way ANOVA, *p-values on the figures). FIG. 2F is a graph comparing various human breast cancer cell line growth rates. FIG. 2G is a series of immunoblots of pAKT in nuclear fractions of Pten^{-/-} and Pik3ca mutant MEFs. FIG. 2H depicts the confluence after 5 days of cells treated with 50 μM leflunomide in combination with 0 or 640 μM orotate (Student's t-test, *p<0.05, n=3). FIG. 2I depicts the confluence after 5 days of cells treated with 50 μM leflunomide in combination with 0, 31.25, 62.5, or 125 μM orotate (Student's t-test, *p<0.05, n=3). Data shown as mean+SD.

FIG. 3A is a graph showing the GI50 of human breast, glioblastoma, and prostate cell lines treated with eight doses of leflunomide (Student's t-test, *p<0.05, n=3). FIG. 3B is a graph showing the tumor volume of PTEN-mutant triple-negative breast cancer cell line SUM149 xenografts. Mice were treated with 100 mg/kg oral leflunomide or vehicle control on days indicated with arrows, and tumor volume was measured by calipers (one-way ANOVA with multiple t-tests, corrected for multiple comparisons, *p<0.01 for ANOVA and t-tests, n=6). FIG. 3C is a graph showing the relative luminescence of PTEN-mutant triple-negative breast cancer cell line MDA-MB 468 xenografts expressing luciferase. Treatment was started on day 7, with 100 mg/kg leflunomide or vehicle control administered orally for four consecutive days each week (one-way ANOVA with multiple t-tests, corrected for multiple comparisons, *p<0.05 for ANOVA and t-tests, n=5). Radiance was measured on days 0, 7, 14, and 21, quantified as photons/second/cm²/steradian. FIG. 3D: luminescence of treated and control mice after 2 weeks of treatment. Data shown as mean+SD for FIG. 3A and +SEM for FIGS. 3B and 3C.

FIG. 4A is a graph showing positively-stained cells labeled with a gamma-H2AX antibody; cells were analyzed by flow cytometry (Student's t-test, *p<0.05, n=3). FIG. 4B is a graph showing cells treated with 100 μM leflunomide for 48 h and labeled with a gamma-H2AX antibody. The mean fluorescence intensity (MFI) was determined using flow cytometry (Student's t-test, *p<0.01, n=3). FIG. 4C is a series of representative photographs showing MEFs treated with 150 μM A771726 for 24 h and labeled with EdU and gamma-H2AX. Left: representative confocal microscopy images. Right: quantified EdU and gamma-H2AX colocalized foci (Student's t-test, *p<0.05, n=3). FIG. 4D is a series of representative photographs showing MEFs treated with 100 μM leflunomide or control for 48 h and labeled with EdU. Left: representative confocal microscopy images. Right: quantification of the number of foci per cell (Student's t-test, p>0.05, n=6). FIG. 4E is a graph showing the percentage of cells treated with 150 μM A771726 for times indicated and labeled with antibodies to RPA and gamma-H2AX positively staining for RPA alone or both RPA and gamma-H2AX; cells were analyzed by flow cytometry (Student's t-test, *p<0.05, n=4). FIG. 4F is a series of immunoblots of pChk1 after 150 μM A771726 treatment for times indicated. FIGS. 4G and 4H are graphs quantifying the number of chromosomal breaks and multiradial formations per haploid genome (Student's t-test, *p-values on figure, cells scored/replicate>100). Data shown as mean+SD.

FIG. 5A is an immunoblot of PTEN protein of MEFs derived from two independent embryos, infected with an empty adenovirus or one containing cre recombinase, 2 passages after infection. FIG. 5B depicts the confluence of WT MEFs (with no loxP sites) infected with an empty adenovirus or one containing cre recombinase to determine whether Cre alone affects growth (Student's t-test, p>0.05, non-significant, n=3). FIG. 5C is a graph showing positively-stained cells labeled with annexin V and 7AAD; cells were analyzed by flow cytometry (Student's t-test, p>0.05, n=3). FIG. 5D is a graph showing positively-stained cells labeled with BrdU followed by an anti-BrdU antibody and propidium iodide; cells were analyzed by flow cytometry to determine the cells in each population corresponding to G1, S, and G2 phases of the cell cycle (Student's t-test, *p<0.001, n=3). FIG. 5E depicts the confluence of WT MEFs grown in the presence of full glucose or no added glucose. Data shown as mean+SD.

FIG. 6A is a table showing data from over 200 metabolites measured by LC-MS/MS from unlabeled MEFs. Data were analyzed with the Integrated Molecular Pathway Analysis program (IMPaLA) and the top 5 hits for pathways upregulated in Pten−/− MEFs are shown in green, all related to pyrimidine metabolism. As a comparison, 5 other pathways upregulated in Pten−/− MEFs are shown: purine metabolism, the TCA cycle, and glucose metabolism are farther down the list. FIG. 6B is a series of bar graphs showing the relative levels of each metabolite listed in the “pyrimidine metabolism” and “nucleotide metabolism” pathways from FIG. 6A. (Student's t-test, *p<0.05, n=3). FIG. 6C shows the metabolites from FIG. 1D mapped out onto the de novo pyrimidine synthesis pathway. Graphs on the left side of the figure correspond to 15N labeled glutamine, and on the right side to 13C labeled glutamine. Some metabolites are missing either a 13C or 15N graph; not every metabolite was able to be measured in both conditions (Student's t-test, *p<0.05, n=3). FIG. 6D shows a gene set enrichment analysis of the pyrimidine synthesis gene set on microarray data from MEFs (FDR q-value<0.05).

FIG. 7A depicts the confluence of Pten WT or KO MEFs incubated with 25 μM leflunomide. FIG. 7B depicts the confluence of Pten WT or KO MEFs incubated with 100 μM leflunomide (one-way ANOVA, *p<0.001, n=3). FIG. 7C, FIG. 7D, and FIG. 7E are series of bar graphs depicting the GI50s of cells treated with dose titrations of DHODH inhibitors as indicated (Student's t-test, *p-values as reported on the figures, n=3). FIG. 7F shows the accumulation of cell death overtime as determined by live cell imaging (6 h intervals). Cells were treated with 100 μM leflunomide and DRAQ7 (one-way ANOVA between PTEN WT and mut, *p<0.01). FIG. 7G depicts the confluence of MCCL-278 and MCCL-357 breast cells. FIG. 7H depicts the confluence of Myc-CaP and CaP8 prostate cells. FIG. 7I is an immunoblot of pAKT in MCCL-278 and MCCL-357 breast cells. FIG. 7J is an immunoblot of pAKT in nuclear fractions of MCCL-278 and MCCL-357 breast cells. Data shown as means±SD.

FIG. 8A is a bar graph showing the confluence after 5 days of cells treated with 25 μM leflunomide in combination with 0, 312.5, or 625 μM orotate (Student's t-test, *p<0.05, n=3).

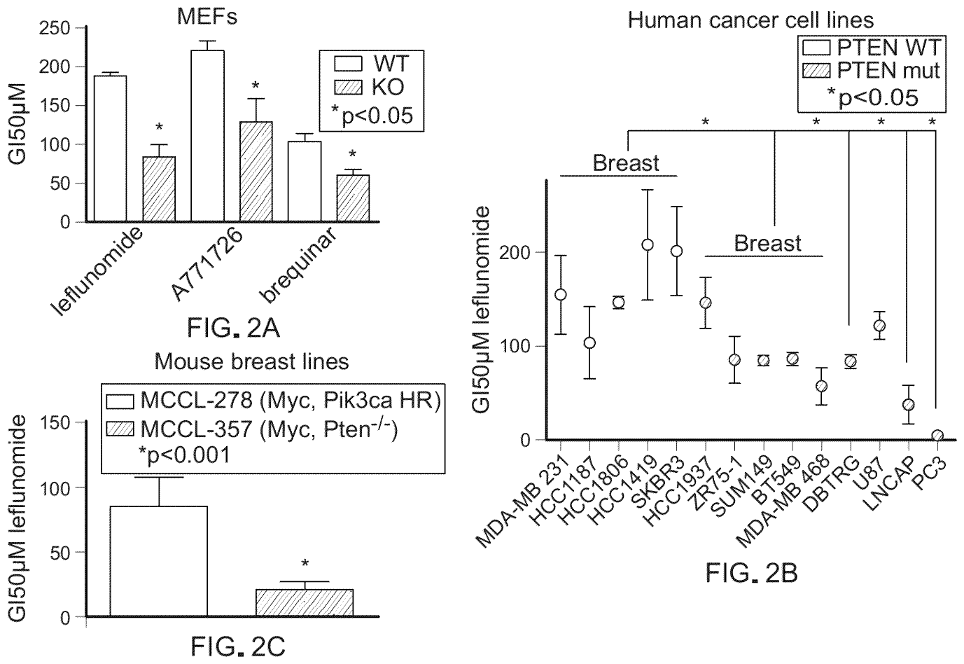

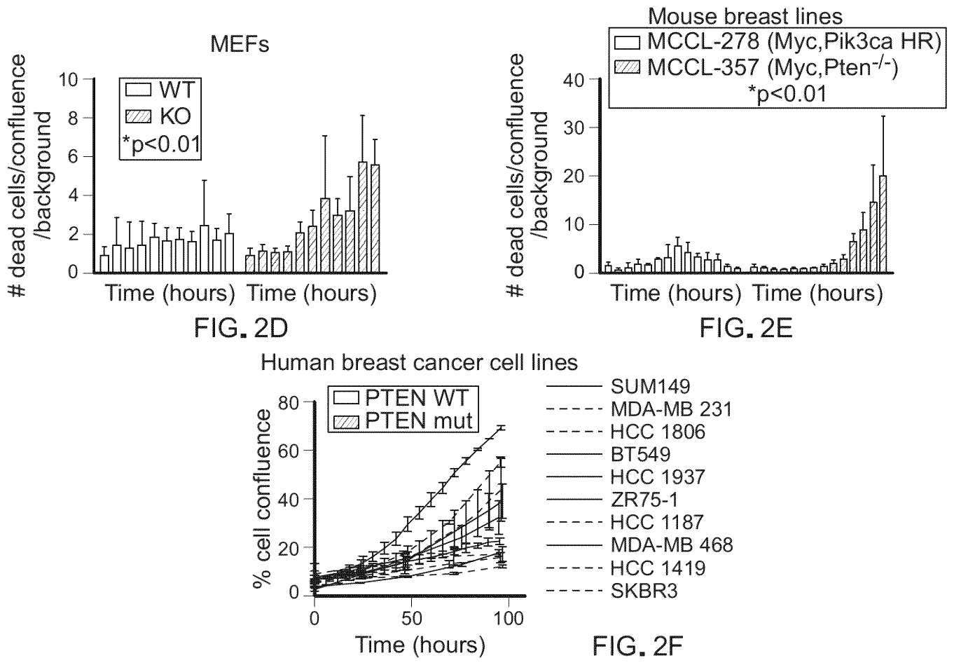

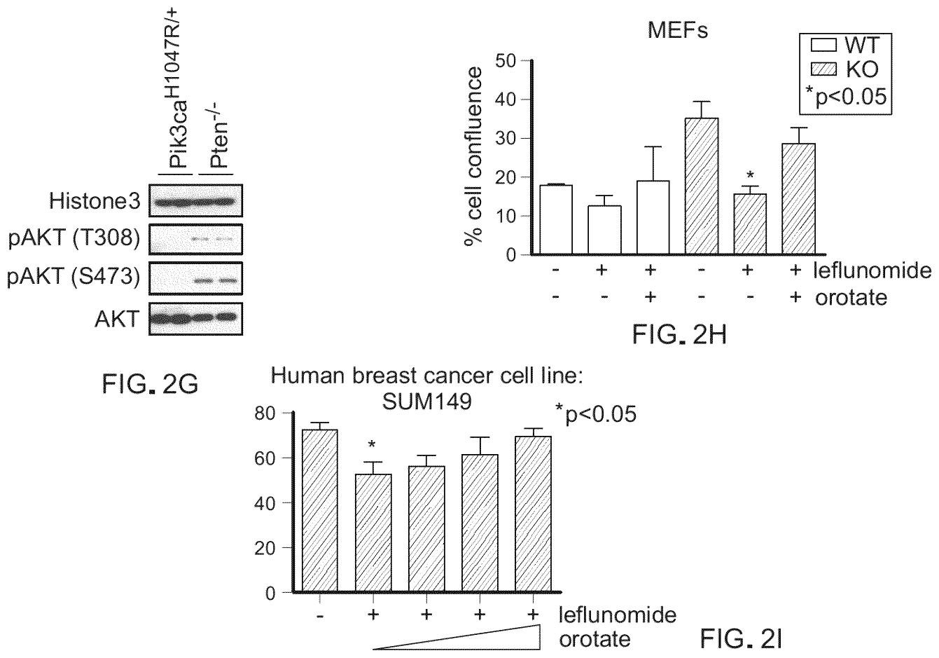

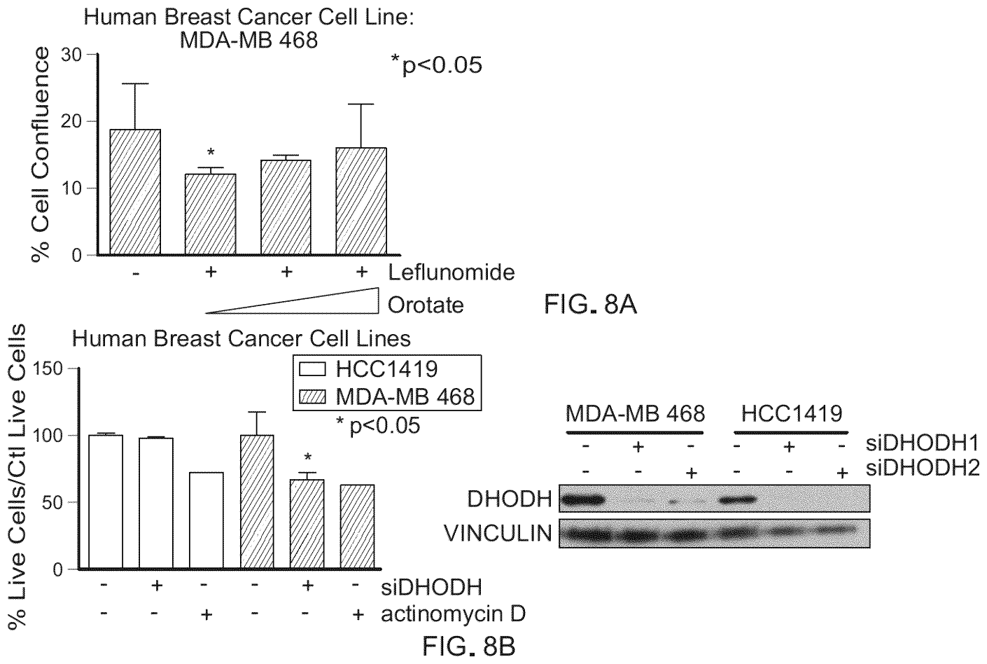

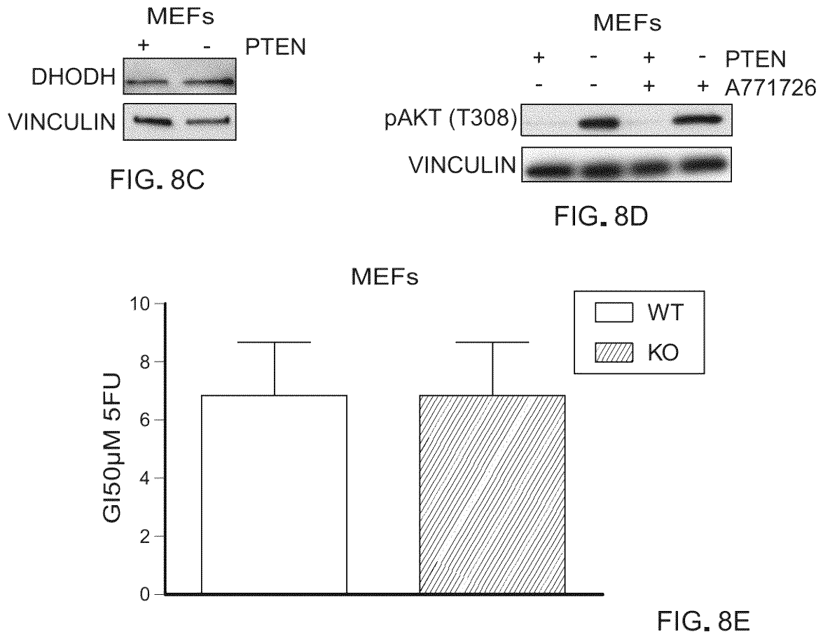

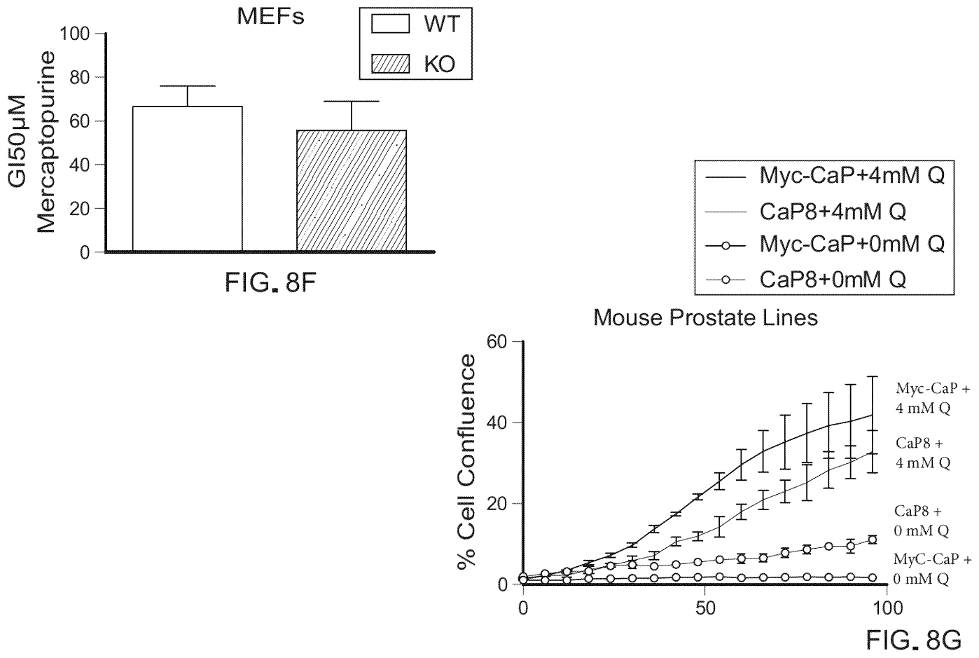

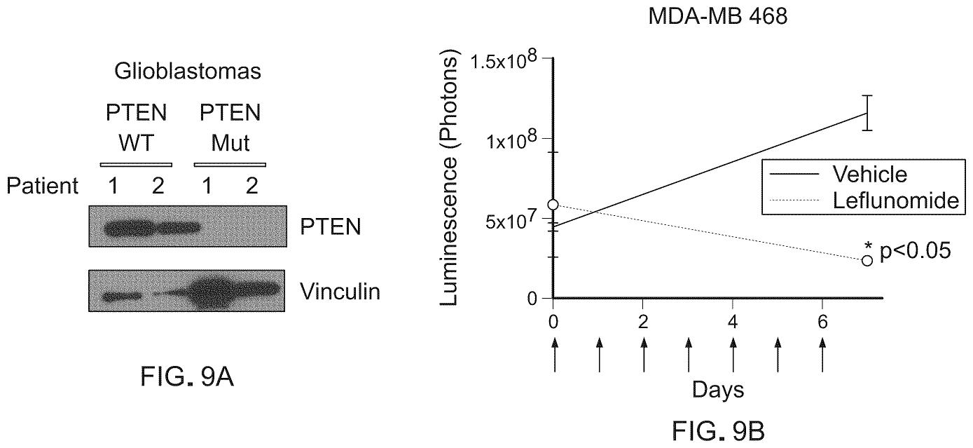

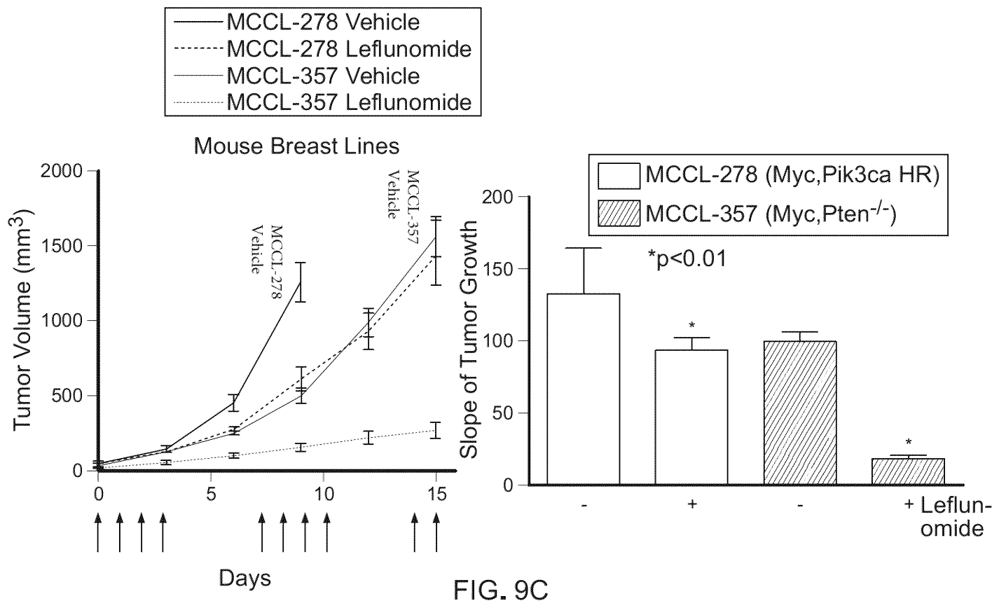

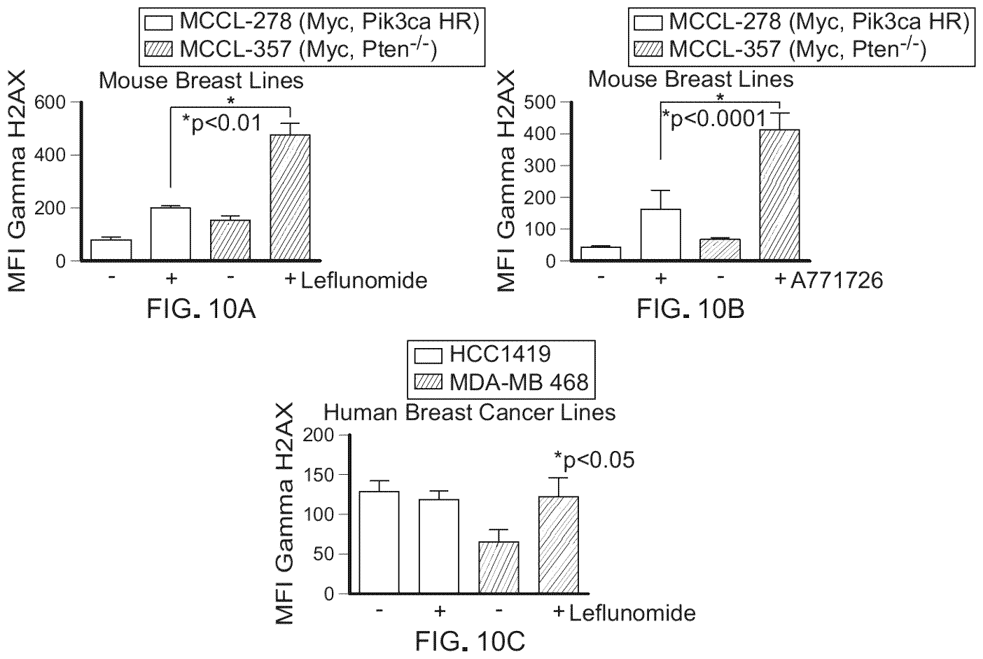

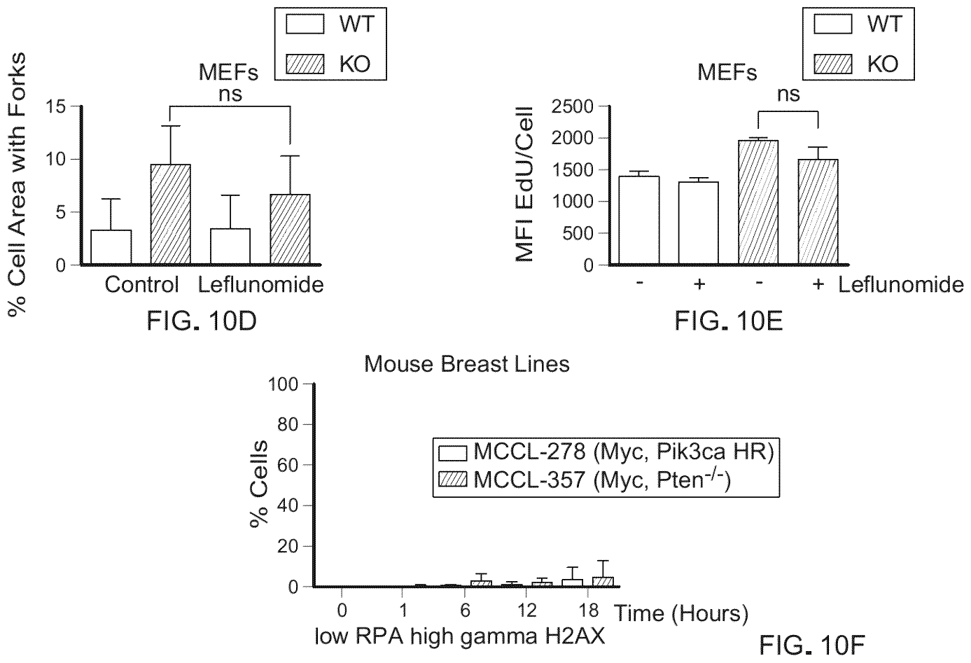

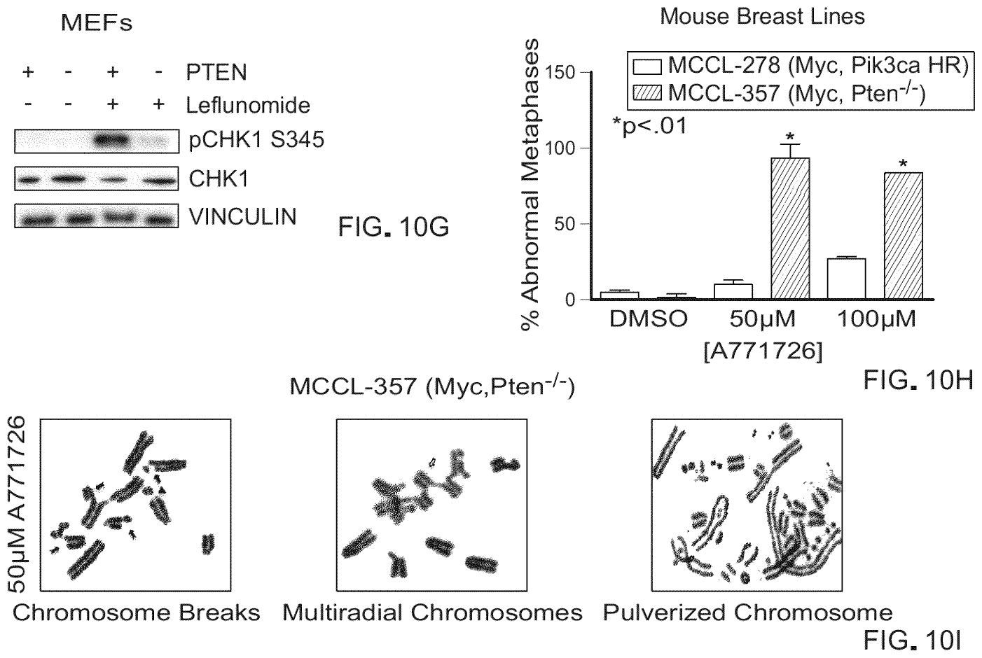

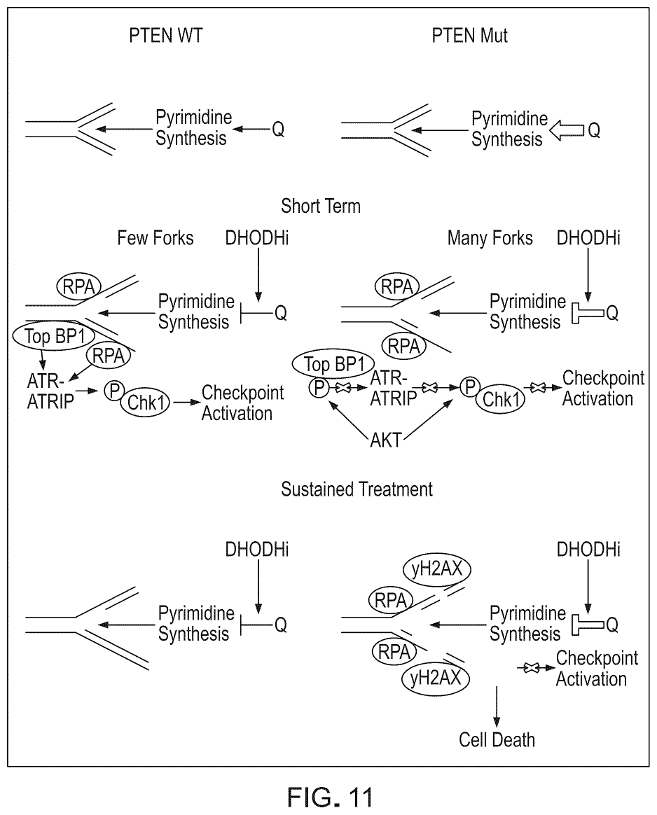

Note that here as well as in FIG. 2, H-I, a large amount of DMSO was used in each condition to match the amount of orotate needed, narrowing the growth differential normally observed between leflunomide-treated and untreated cells in the PTEN mutant setting. FIG. 8B is a bar graph and immunoblot depicting cells transfected with siRNA against DHODH. The bar graph depicts cell viability measured using annexin V and 7AAD. 0.5 μg/mL actinomycin D was a positive control for cell death. (Students t-test, *p<0.05, n=3). The immunoblot shows DHODH after knockdown with one of two DHODH siRNAs or a control siRNA. FIG. 8C is an immunoblot of DHODH in Pten KO and WT cells. FIG. 8D is an immunoblot of pAKT in Pten KO and WT cells with or without treatment with 50 μM A771726. FIG. 8E and FIG. 8F are bar graphs depicting the GI50s of cells treated with dose titrations of 5-fluorouracil or mercaptopurine, respectively (Student's t-test, p>0.05, n=3). FIG. 8G depicts the confluence of prostate cells grown in media containing full glutamine (4 mM) or no added glutamine. Data shown as means±SD.

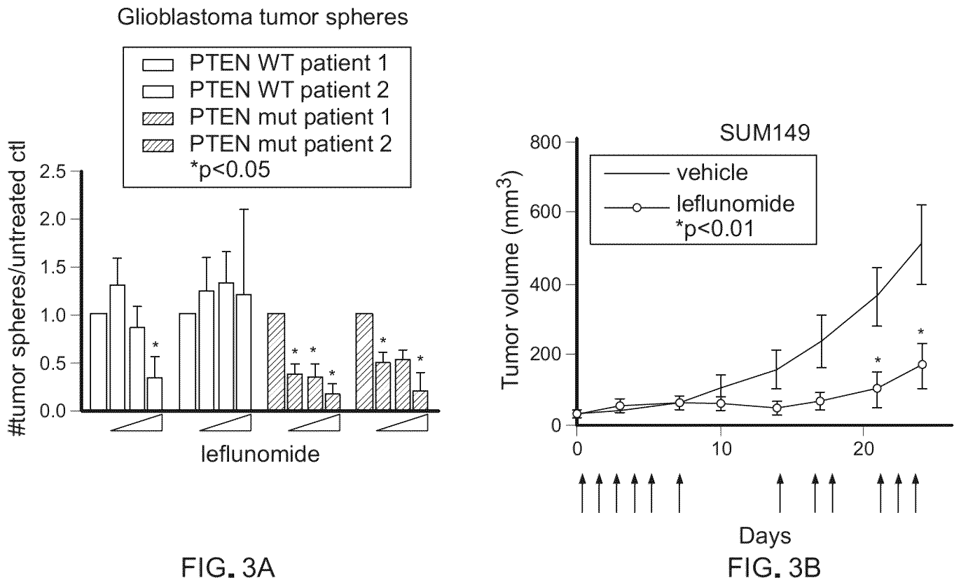

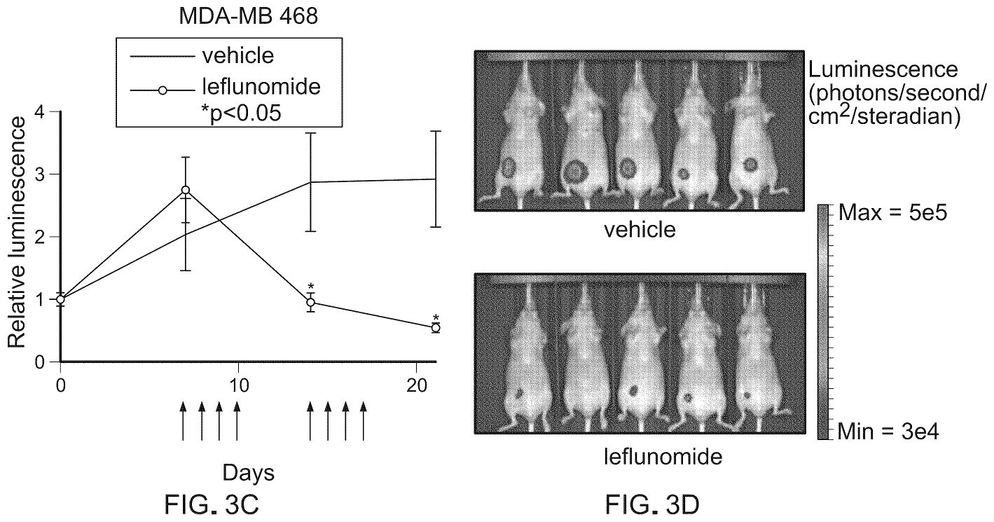

FIG. 9A is an immunoblot of PTEN in the four patient-derived glioblastomas in FIG. 3A. FIG. 9B is a graph showing the size of MDA-MB 468 xenografts which were never treated and allowed to grow for seven weeks, then treated with vehicle or 100 mg/kg leflunomide for 7 days. Tumor size was measured by assessing luminescence, quantified by photons/second/cm²/steradian (n=2). FIG. 9C is a graph showing the size of MCCL-278 (Myc, Pik3ca HR) and MCCL-357 (Myc, Pten−/−) xenografts which were treated with vehicle or 100 mg/kg leflunomide for four consecutive days each week. Tumor volume was measured by calipers. Growth rate of the tumors was determined by calculating the slope of the tumor growth (Student's t-test, *p<0.01, n=8). Data shown as means±SEM.

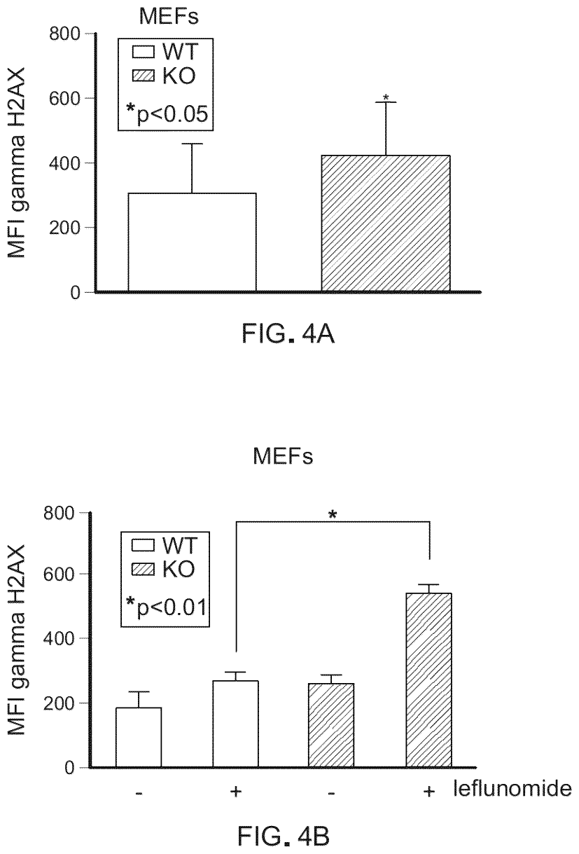

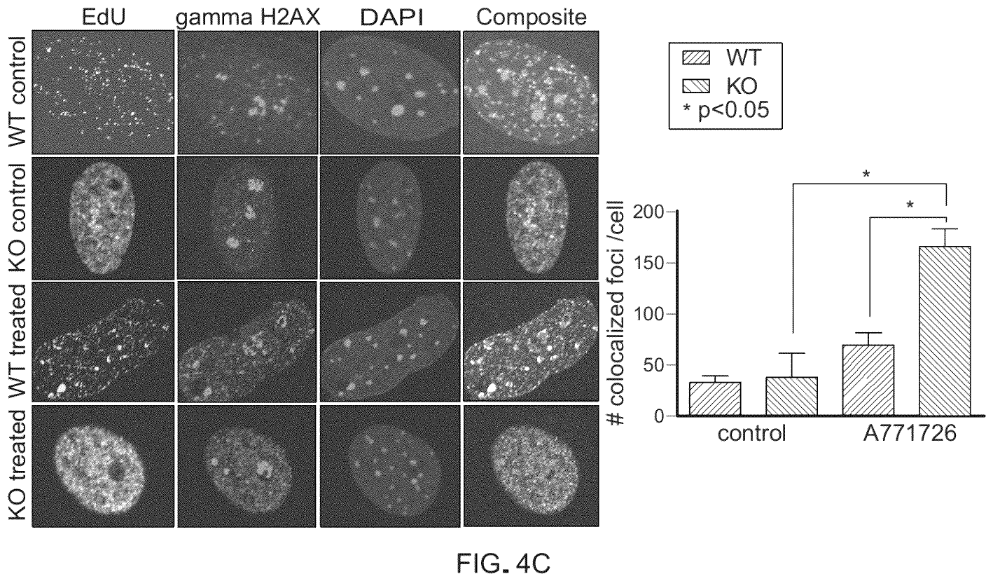

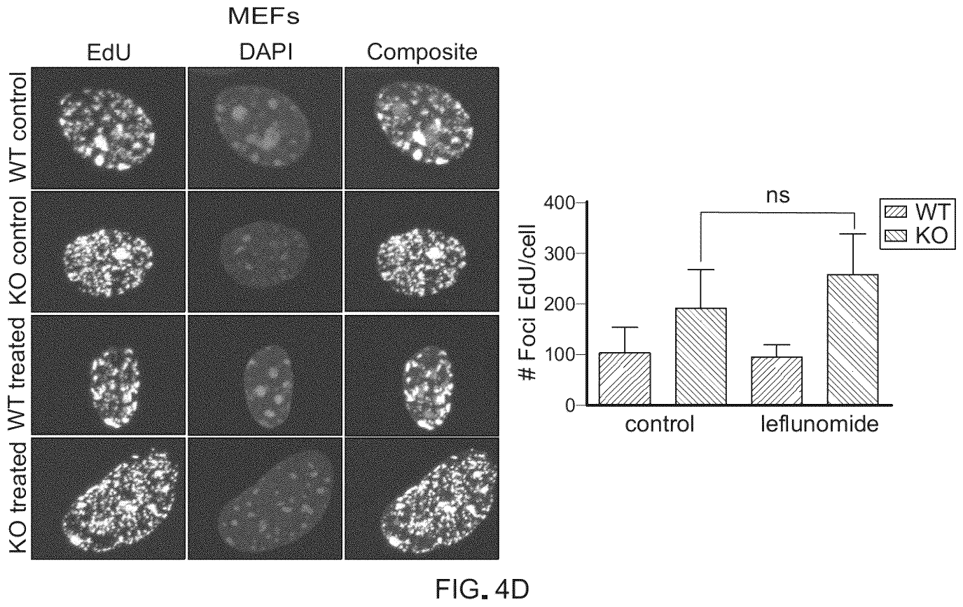

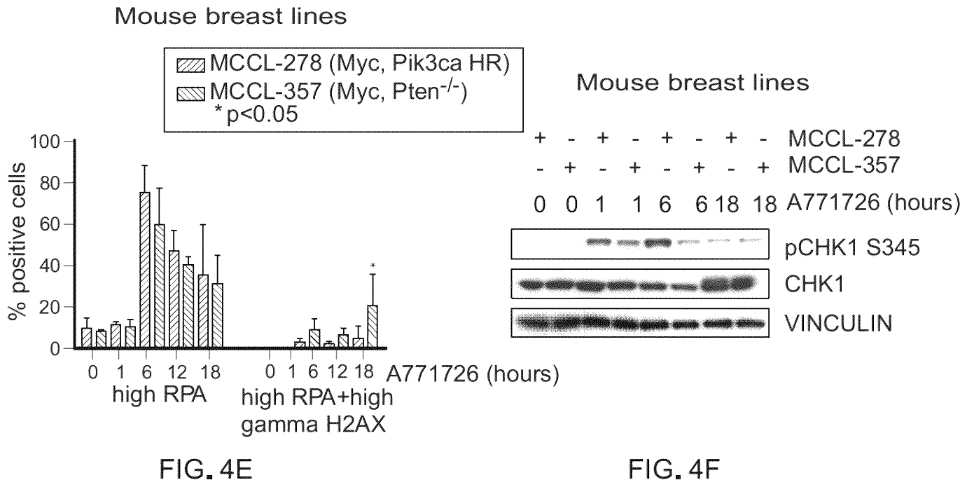

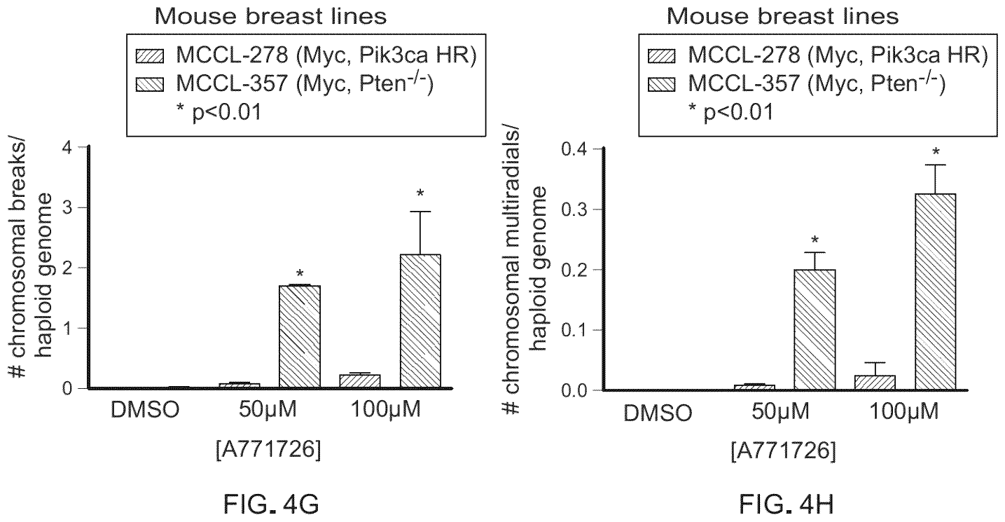

FIG. 10A, FIG. 10B, and FIG. 10C are bar graphs showing quantitative depictions of the mean fluorescence intensity of human or mouse breast cells treated with leflunomide or A771726 labeled with a gamma-H2AX antibody; cells were analyzed by flow cytometry (Student's t-test, *p-values on figures, n=3). FIG. 10D is a bar graph showing a quantitative depiction of the percent cell area with EdU-staining from images in FIG. 4D, used to normalize foci to cell size (Student's t-test, p>0.05, not significant, n=6). FIG. 10E is a bar graph showing a quantitative depiction of the mean fluorescence intensity of Pten WT and KO MEFs treated with 100 μM leflunomide or control for 48 h and labeled with EdU for 45 min; cells were analyzed by flow cytometry (Student's t-test, p>0.05, n=3). FIG. 10F is a bar graph showing the percentage of MCCL-278 and MCCL-357 breast cells positively stained for gamma-H2AX and negatively stained for RPA. Cells were treated with A771726 for times indicated and co-stained with antibodies to RPA and gamma-H2AX; cells were analyzed by flow cytometry (n=4). FIG. 10G is an immunoblot of pChk1 in Pten KO and WT MEFs with or without treatment with 200 μM leflunomide for 24 h. FIG. 10H is a bar graph showing a quantitative depiction of the percent of abnormal metastases in MCCL-278 and MCCL-357 breast cells treated with 50 or 100 μM A771726 or vehicle (Student's t-test, *p<0.01). FIG. 10I is a series of representative images of the types of DNA damage accrued in MCCL-357 cells treated with 50 μM A771726 for 48 hours. Pulverized chromosomes could not be quantified due to the very high number of fragments.

FIG. 11 is a model of WT (left) and Pten−/− cells (right) before and after DHODH inhibition. After glutamine enters Pten−/− cells, it is largely channeled into pyrimidine synthesis to help sustain the greater number of replication forks relative to WT cells. DHODH inhibition blocks pyrimidine synthesis, leading to stalled forks and RPA loading. In the setting of PTEN deficiency, AKT phosphorylates Chk1 and TopBP1, releasing TopBP1 from chromatin and preventing checkpoint activation. Cells continue to attempt division while DNA damage accumulates, leading to cell death. WT cells do not have the same dependency on glutamine flux into pyrimidine synthesis, high number of replication forks, or inherent Chk1 defects, and therefore do not exhibit the same downstream consequences of DHODH inhibition.

### DETAILED DESCRIPTION

The present disclosure is based, in part, on the discovery that dihydroorotate dehydrogenase (DHODH) inhibitors are useful in the treatment of phosphatase and tensin homolog (PTEN)-mutant cancer. As discussed in the following examples, this disclosure examined the metabolic consequences of PTEN mutation (e.g., resulting in partial or complete PTEN inactivation or deficiency that occurs during tumor development) and identified the resulting vulnerability of PTEN-mutant (e.g., PTEN-deficient/negative) tumors. PTEN mutation leads to, among other effects, chemoresistance in prostate cancer, a poorer response to trastuzumab in triple-negative breast cancer, and a shorter survival time in patients with gliomas. Mutation of PTEN can occur through multiple mechanisms and is herein defined as one or more deletions (ranging in size from 1 bp to entire gene or greater), fusions, missense/nonsense alterations within one or more exons, and/or splice site intronic alterations. Alteration of PTEN can be detected in the germline or within the tumor at different points during tumor progression. Targeting the vulnerabilities resulting from mutation of PTEN can be beneficial, particularly since the standard of care for the aforementioned cancers is primarily chemotherapy and radiation.

Dihydroorotate dehydrogenase (DHODH) is a mitochondrial enzyme which catalyzes the ubiquinone-mediated oxidation of dihydroorotate to orotate, in de novo pyrimidine biosynthesis. Inhibiting both DHODH and tyrosine kinases (e.g., the src-family, Polo-like, platelet derived growth factor receptor, epidermal growth factor receptor and fibroblast growth factor receptor arrests lymphocytes in G1, leading to anti-inflammatory and immunomodulatory effects, including decreased expression of adhesion molecules, metalloproteinases, IL-2, IL-6, IL-10, NF-κB, cyclooxygenases, TGF-β1, CD4 T cells, and dendritic cells.

An exemplary DHODH inhibitor, leflunomide is an oral pro-drug that is metabolized by the gut and liver to teriflunomide (also known as A771726 or (Z)-2-Cyano-3-hydroxy-but-2-enoic acid-(4trifluoromethylphenyl)-amide, empirical formula C₁₂H₉F₃N₂O₂, molecular weight 270.21) and has been used in human patients to treat rheumatoid arthritis (RA), psoriatic arthritis, Wegner's granulomatosis, for post-transplant immunosuppression and polyomavirus-induced allograft.

Inhibiting DHODH has the advantage of affecting a specific pathway of glutamine flux downstream of glutaminase, thus preserving glutamine's other important functions in the cell. This increases the specificity of DHODH inhibitors to cells that are dependent on glutamine's role in pyrimidine synthesis per se, and (without wishing to be bound by theory) is perhaps why their toxicity is low enough to allow daily administration to patients to treat other conditions (e.g., rheumatoid arthritis or multiple sclerosis). Activation of mTORC1, which occurs as a consequence of PTEN homozygous deletion, increases glutamine flux into the de novo pyrimidine synthesis pathway through regulation of CAD, a key enzyme that generates dihydroorotate. High activation of AKT toward TOPBP1 and CHK1 that down regulate ATR activation at replication forks compounded with enhanced pyrimidine flux that both occur as a consequence of PTEN inactivation can create synthetic lethality between PTEN mutation and DHODH inhibition. That is, PTEN-mutant tumor cells exhibit a strong tendency to be more sensitive to growth inhibition by DHODH inhibitors (e.g., leflunomide) than PTEN WT tumor cells.

Thus, DHODH inhibitors provide a targeted therapy for patients with PTEN-mutant cancers. As shown in the Examples below, exemplary DHODH inhibitors have demonstrated efficacy (as evidenced by, e.g., changes in glutamine metabolism, DNA replication, and/or DNA damage response) both in vitro and in vivo in treating PTEN-mutant tumors derived from different tissues.

The examples below show that increased growth of PTEN-mutant cells is dependent on glutamine flux through the de novo pyrimidine synthesis pathway, which creates sensitivity to inhibition of dihydroorotate dehydrogenase (DHODH), a rate-limiting enzyme for pyrimidine ring synthesis. S-phase PTEN-mutant cells show increased numbers of replication forks, and inhibitors of dihydroorotate dehydrogenase cause chromosome breaks and cell death due to inadequate ATR activation and DNA damage at replication forks. Without wishing to be bound by theory, these findings indicate that enhanced glutamine flux generates vulnerability to dihydroorotate dehydrogenase inhibition, which then causes synthetic lethality in PTEN-mutant cells due to inherent defects in ATR activation.

Further, without wishing to be bound by theory, the experiments indicate that inhibition of DHODH in PTEN-mutant cells first causes stalled forks due to inadequate nucleotide pools required to support replication; sustained treatment leads to insufficient ATR activation due to AKT phosphorylation of TOPBP1 and CHK1, leading to a buildup of DNA damage and cell death associated with mitotic catastrophe. PTEN wild-type (WT) cells do not exhibit this dependency on pyrimidine synthesis and have fewer forks per cell, perhaps because ATR-CHK1 coordinates origin firing during S-phase. In PTEN WT cells, treatment initially increased the RPA signal and triggered transient phosphorylation of CHK1, but longer treatment led to abated RPA with little concurrent increase in gamma-H2AX, explaining the largely unaffected WT population upon DHODH inhibition (FIG. 11). While Pik3ca mutant cells also exhibit AKT signaling, their relative resistance to DHODH inhibitors indicates that a dosage effect due to their lower level of AKT activation may be important.

### Teriflunomide

Teriflunomide is the principal active metabolite of leflunomide and is responsible for leflunomide's activity in vivo. At recommended doses, administration of teriflunomide or leflunomide to a patient result in a similar range of plasma concentration of teriflunomide. Based on a population analysis of teriflunomide in healthy volunteers and MS patients, median t½ was approximately 18 and 19 days after repeated doses of 7 mg and 14 mg, respectively. It takes approximately 3 months respectively to reach steady-state concentrations. The estimated AUC accumulation ratio is approximately 30 after repeated doses of 7 or 14 mg. Median time to reach maximum plasma concentrations is between 1 to 4 hours post-dose following oral administration of teriflunomide. Food does not have a clinically relevant effect on teriflunomide pharmacokinetics. Teriflunomide has a low volume of distribution (Vss=0.13 L/kg) and is extensively bound (>99.3%) to albumin in healthy subjects. Protein binding has been shown to be linear at therapeutic concentrations. The free fraction of teriflunomide is slightly higher in patients with rheumatoid arthritis and approximately doubled in patients with chronic renal failure; the mechanism and significance of these increases are unknown. Teriflunomide is the major circulating moiety detected in plasma. The primary biotransformation pathway to minor metabolites of teriflunomide is hydrolysis, with oxidation being a minor pathway. Secondary pathways involve oxidation, N-acetylation and sulfate conjugation.

Teriflunomide is eliminated mainly through direct biliary excretion of unchanged drug as well as renal excretion of metabolites. Over 21 days, 60.1% of the administered dose is excreted via feces (37.5%) and urine (22.6%). After an accelerated elimination procedure with cholestyramine, an additional 23.1% is eliminated (mostly in feces). After a single IV administration, the total body clearance of teriflunomide is 30.5 mL/h. Teriflunomide is eliminated slowly from the plasma. Without an accelerated elimination procedure, it takes on average 8 months to reach plasma concentrations less than 0.02 mg/L, although because of individual variations in drug clearance it can take as long as 2 years. An accelerated elimination procedure could be used at any time after discontinuation of teriflunomide or leflunomide. Elimination can be accelerated, e.g., by either of the following procedures:

Administration of cholestyramine 8 g every 8 hours for 11 days. If cholestyramine 8 g three times a day is not well tolerated, cholestyramine 4 g three times a day can be used.

Administration of 50 g oral activated charcoal powder every 12 hours for 11 days.

If either elimination procedure is poorly tolerated, treatment days do not need to be consecutive unless there is a need to lower teriflunomide plasma concentration rapidly. At the end of 11 days, both regimens successfully accelerate teriflunomide elimination, leading to a more than 98% decrease in teriflunomide plasma concentration.

A population-based pharmacokinetic analysis of teriflunomide's phase III data indicates that smokers have a 38% increase in clearance over non-smokers; however, no difference in clinical efficacy was seen between smokers and nonsmokers. In a population analysis, the clearance rate for teriflunomide is 23% less in females than in males. In single-dose studies in patients (n=6) with chronic renal insufficiency requiring either chronic ambulatory peritoneal dialysis (CAPD) or hemodialysis, neither had a significant impact on circulating levels of teriflunomide. The free fraction of teriflunomide was almost doubled, but the mechanism of this increase is not known. In light of the fact that the kidney plays a role in drug elimination and without adequate studies of leflunomide use in subjects with renal insufficiency, caution should be used when leflunomide is administered to these patients. Given the need to metabolize leflunomide into the active species, the role of the liver in drug elimination/recycling, and the possible risk of increased hepatic toxicity, the use of leflunomide in patients with hepatic insufficiency is not recommended. Teriflunomide is pregnancy category X (unsafe). It should not be administered to nursing mothers. In a placebo controlled thorough electrocardiogram QT study performed in healthy subjects, there was no evidence that teriflunomide caused QT interval prolongation of clinical significance (i.e., the upper bound of the 90% confidence interval for the largest placebo-adjusted, baseline-corrected QTc was below 10 ms).

There is an increase in mean repaglinide Cₘₐₓ and AUC (1.7- and 2.4-fold, respectively) following repeated doses of teriflunomide and a single dose of 0.25 mg repaglinide, suggesting that teriflunomide is an inhibitor of CYP2C8 in vivo. The magnitude of interaction could be higher at the recommended repaglinide dose. Repeated doses of teriflunomide decrease mean Cₘₐₓ and AUC of caffeine by 18% and 55%, respectively, suggesting that teriflunomide may be a weak inducer of CYP1A2 in vivo. There is an increase in mean cefaclor Cₘₐₓ and AUC (1.43- and 1.54-fold, respectively), following repeated doses of teriflunomide, suggesting that teriflunomide is an inhibitor of organic anion transporter 3 (OAT3) in vivo. There is an increase in mean rosuvastatin Cₘₐₓ and AUC (2.65- and 2.51-fold, respectively) following repeated doses of teriflunomide, suggesting that teriflunomide is an inhibitor of BCRP transporter and organic anion transporting polypeptide 1B1 and 1B3 (OATP1B1/1B3). There is an increase in mean ethinylestradiol Cₘₐₓ and AUC 0-24 (1.58- and 1.54-fold, respectively) and levonorgestrel Cₘₐₓ and AUC 0-24 (1.33- and 1.41-fold, respectively) (in other words, elevated levels of these estrogens) following repeated doses of teriflunomide. Teriflunomide does not affect the pharmacokinetics of bupropion (a CYP2B6 substrate), midazolam (a CYP3A4 substrate), S-warfarin (a CYP2C9 substrate), omeprazole (a CYP2C19 substrate), or metoprolol (a CYP2D6 substrate). Rifampin does not affect the pharmacokinetics of teriflunomide.

The immunomodulatory agent teriflunomide, clinically FDA-approved for multiple sclerosis, has been shown to have anti-inflammatory properties. The drug has been given to over 2000+ patients in published literature studies alone, and its pharmacokinetics, pharmacodynamics, oral bioavailability, half-life, metabolism, protein binding, and side effects are well-described (see, e.g., Table 1 below).

TABLE 1
Side effects occurring reported for at least 2% of patients 7 mg/day or
14 mg/day of teriflunomide and 2% above placebo. These data are based
on multiple sclerosis patients, 71% female with mean age of 37 years.

| | 7 mg | 14 mg | Placebo |
|---|---|---|---|
| Side effects* | (n = 1045) | (n = 1002) | (n = 997) |
| Neutropenia | 4 | 6 | 2 |
| Nausea | 8 | 11 | 7 |
| Diarrhea | 13 | 14 | 8 |
| Elevated ALT^{§} | 6 | 6 | 4 |
| Arthralgia | 8 | 6 | 5 |
| Alopecia | 10 | 13 | 5 |
| Headache | 18 | 16 | 15 |
| Parathesia | 8 | 9 | 7 |
| Serious Infection | 2 | 3 | 2 |
| Hypertension (new onset) | 3 | 4 | 2 |
| Peripheral Neuropathy^{¶} | 0.3 | 0.5 | — |
| Elevated ALT^{¶} | 3 | 3 | 2 |

^{§}>3 × upper limit of normal
^{¶}Serious adverse events leading to treatment withdrawal
*All grades of side effects

In certain aspects, the methods described herein include the manufacture and use of pharmaceutical compositions and medicaments that include compounds identified by a method described herein as active ingredients. Also included are the pharmaceutical compositions themselves.

In some instances, the compositions disclosed herein can include other compounds, drugs, and/or agents used for the treatment of cancer. For example, in some instances, therapeutic compositions disclosed herein can be combined with one or more (e.g., one, two, three, four, five, or less than ten) compounds.

In some instances, the compositions disclosed herein can include DHODH inhibitors (e.g., DHODH selective inhibitor) such as, for example brequinar, leflunomide, redoxal, S-2678, or teriflunomide.

A DHODH inhibitor may selectively affect PTEN-mutant compared to PTEN WT cells (i.e., an inhibitor able to kill or inhibit the growth of a PTEN-mutant cell while also having a relatively low ability to lyse or inhibit the growth of a PTEN WT cell), e.g., possess an IC₅₀ for one or more PTEN-mutant cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its IC₅₀ for one or more PTEN WT cells, e.g., PTEN WT cells of the same species and tissue type as the PTEN-mutant cells.

One or more of the DHODH inhibitors disclosed herein can be formulated for use as or in pharmaceutical compositions. Such compositions can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs).

The pharmaceutical compositions may be formulated for oral, parenteral, or transdermal delivery. The compound of the invention may also be combined with other pharmaceutical agents.

The pharmaceutical compositions disclosed herein can be administered, e.g., orally, parenterally, by inhalation spray or nebulizer, topically, rectally, nasally, buccally, vaginally, via an implanted reservoir, by injection (e.g., intravenously, intra-arterially, subdermally, intraperitoneally, intramuscularly, and/or subcutaneously), in an ophthalmic preparation, or via transmucosal administration. Suitable dosages may range from about 0.001 to about 100 mg/kg of body weight, or according to the requirements of the particular drug. The pharmaceutical compositions of this invention can contain any conventional non-toxic pharmaceutically-acceptable carriers, adjuvants or vehicles. In some cases, the pH of the formulation can be adjusted with pharmaceutically acceptable acids, bases, or buffers to enhance the stability of the formulated compound or its delivery form. The term “parenteral” as used herein includes subcutaneous, intracutaneous, intravenous, intramuscular, intraarticular, intra-arterial, intrasynovial, intrasternal, intrathecal, intralesional, and intracranial injection or infusion techniques. Alternatively or in addition, the present invention may be administered according to any of the methods as described in the FDA DSM.

Pharmaceutical compositions typically include a pharmaceutically acceptable carrier. The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration.

As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. As used herein, the term “pharmaceutically acceptable derivative” means any pharmaceutically acceptable salt, solvate or prodrug, e.g., ester, of an atovaquone-related compound described herein, which upon administration to the recipient is capable of providing (directly or indirectly) a compound described herein, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. Pharmaceutically acceptable derivatives include salts, solvates, esters, carbamates, and/or phosphate esters.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intra-articular, intraarterial, intrasynovial, intrasternal, intrathecal, intralesional and intracranial injection or infusion techniques.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

As used herein, the DHODH inhibitors disclosed herein are defined to include pharmaceutically acceptable derivatives or prodrugs thereof. A “pharmaceutically acceptable derivative or prodrug” means any pharmaceutically acceptable salt, ester, salt of an ester, or other derivative of a compound or agent disclosed herein which, upon administration to a recipient, is capable of providing (directly or indirectly) a compound of this invention. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein.

In some instances, pharmaceutical compositions can include an effective amount of one or more DHODH inhibitors. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cancer).

In some embodiments, the present disclosure provides methods for using a composition comprising a DHODH inhibitor, including pharmaceutical compositions (indicated below as ‘X’) disclosed herein in the following methods:

Substance X for use as a medicament in the treatment of one or more diseases or conditions disclosed herein (e.g., neurodegenerative disease, referred to in the following examples as ‘Y’). Use of substance X for the manufacture of a medicament for the treatment of Y; and substance X for use in the treatment of Y.

In some instances, therapeutic compositions disclosed herein can be formulated for sale in the US, import into the US, and/or export from the US.

The pharmaceutical compositions can be included in a container, pack, or dispenser together with instructions for administration.

The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some embodiments, an effective dose of a DHODH inhibitor can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day.

In some embodiments, an effective dose of teriflunomide can include, e.g., about 0.1, 0.2, 0.3, 0.4, 0.5, 0.6, 0.7, 0.8, 0.9, or 1 mg/kg/day. In some embodiments, an effective dose of teriflunomide can be, e.g., about 0.2 mg/kg/day. In some embodiments, an effective dose of leflunomide can include, e.g., about 0.1, 0.2, 0.3, 0.4, 0.5, 0.6, 0.7, 0.8, 0.9, or 1 mg/kg/day. In some embodiments, an effective dose of leflunomide can be, e.g., about 0.3 mg/kg/day.

Pharmaceutical compositions of this invention can include one or more DHODH inhibitors and any pharmaceutically acceptable carrier and/or vehicle. In some instances, pharmaceuticals can further include one or more additional therapeutic agents in amounts effective for achieving a modulation of disease or disease symptoms. Such additional therapeutic agents may include conventional chemotherapeutic agents known in the art. When co-administered, DHODH inhibitors disclosed herein can operate in conjunction with conventional chemotherapeutic agents to produce mechanistically additive or synergistic therapeutic effects.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Pharmaceutical compositions can be in the form of a solution or powder for injection. Such compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, and/or other similar emulsifying agents or bioavailability enhancers which are commonly used in the manufacture of pharmaceutically acceptable solid, liquid, or other dosage forms may also be used for the purposes of formulation.

Pharmaceutical compositions can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Alternatively or in addition, pharmaceutical compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Pharmaceutically acceptable salts of the DHODH inhibitors of this disclosure include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., alkali metal (e.g., sodium), alkaline earth metal (e.g., magnesium), ammonium and N-(alkyl) 4+ salts. The invention also envisions the quaternization of any basic nitrogen-containing groups of the inhibitors disclosed herein. Water or oil-soluble or dispersible products can be obtained by such quaternization.

The methods described herein include methods for the treatment of disorders associated with PTEN-mutant cancer, the methods include administering a therapeutically effective amount of a DHODH inhibitor as described herein, to a subject (e.g., a mammalian subject, e.g., a human subject) who is in need of, or who has been determined to be in need of, such treatment.

In some instances, methods can include selection of a human subject who has or had a condition or disease. In some instances, suitable subjects include, for example, subjects who have or had a condition or disease but that resolved the disease or an aspect thereof, present reduced symptoms of disease (e.g., relative to other subjects (e.g., the majority of subjects) with the same condition or disease), and/or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease).

The terms “treat”, “treating” or “treatment” as used herein, refers to partially or completely alleviating, inhibiting, ameliorating, and/or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; and/or reductions in one or more symptoms associated with one or more tumors in a subject relative to the subject's symptoms prior to treatment.

As used herein, the term “treating cancer” means causing a partial or complete decrease in the rate of growth of a tumor, and/or in the size of the tumor and/or in the rate of local or distant tumor metastasis, and/or the overall tumor burden in a subject, and/or any decrease in tumor survival, in the presence of an inhibitor (e.g., a DHODH inhibitor) described herein.

As used herein, the term “preventing a disease” (e.g., preventing cancer) in a subject means for example, to stop the development of one or more symptoms of a disease in a subject before they occur or are detectable, e.g., by the patient or the patient's doctor. Preferably, the disease (e.g., cancer) does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than that which would have occurred without the present invention.

The term “subject,” as used herein, refers to any animal. In some instances, the subject is a mammal. In some instances, the term “subject”, as used herein, refers to a human (e.g., a man, a woman, or a child).

In some instances, subject selection can include obtaining a sample from a subject (e.g., a candidate subject) and testing the sample for an indication that the subject is suitable for selection. In some instances, the subject can be confirmed or identified, e.g. by a health care professional, as having had or having a condition or disease. In some instances, exhibition of a positive immune response towards a condition or disease can be made from patient records, family history, and/or detecting an indication of a positive immune response. In some instances multiple parties can be included in subject selection. For example, a first party can obtain a sample from a candidate subject and a second party can test the sample. In some instances, subjects can be selected and/or referred by a medical practitioner (e.g., a general practitioner). In some instances, subject selection can include obtaining a sample from a selected subject and storing the sample and/or using the in the methods disclosed herein. Samples can include, for example, cells or populations of cells.

In general, methods include selecting a subject and administering to the subject an effective amount of one or more of the DHODH inhibitors described herein, e.g., in or as a pharmaceutical composition, and optionally repeating administration as required for the prophylaxis or treatment of cancer and can be administered, e.g., orally, intravenously or topically.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

In some instances, treatments methods can include a single administration, multiple administrations, and repeating administration as required for the prophylaxis or treatment of the disease or condition from which the subject is suffering (e.g., a PTEN-mutant cancer). In some instances treatment methods can include assessing a level of disease in the subject prior to treatment, during treatment, and/or after treatment. In some instances, treatment can continue until a decrease in the level of disease in the subject is detected.

The terms “administer,” “administering,” or “administration,” as used herein refers to implanting, absorbing, ingesting, injecting, or inhaling, the inventive drug, regardless of form. In some instances, one or more of the compounds disclosed herein can be administered to a subject topically (e.g., nasally) and/or orally. For example, the methods herein include administration of an effective amount of compound or compound composition to achieve the desired or stated effect. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Following administration, the subject can be evaluated to detect, assess, or determine their level of disease. In some instances, treatment can continue until a change (e.g., reduction) in the level of disease in the subject is detected.

Upon improvement of a patient's condition (e.g., a change (e.g., decrease) in the level of disease in the subject), a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of the therapeutic compounds described herein can include a single treatment or a series of treatments. For example, effective amounts can be administered at least once. Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

### EXAMPLES

### Example 1: Relationship Between PTEN, Cell Growth, and Cellular Metabolism

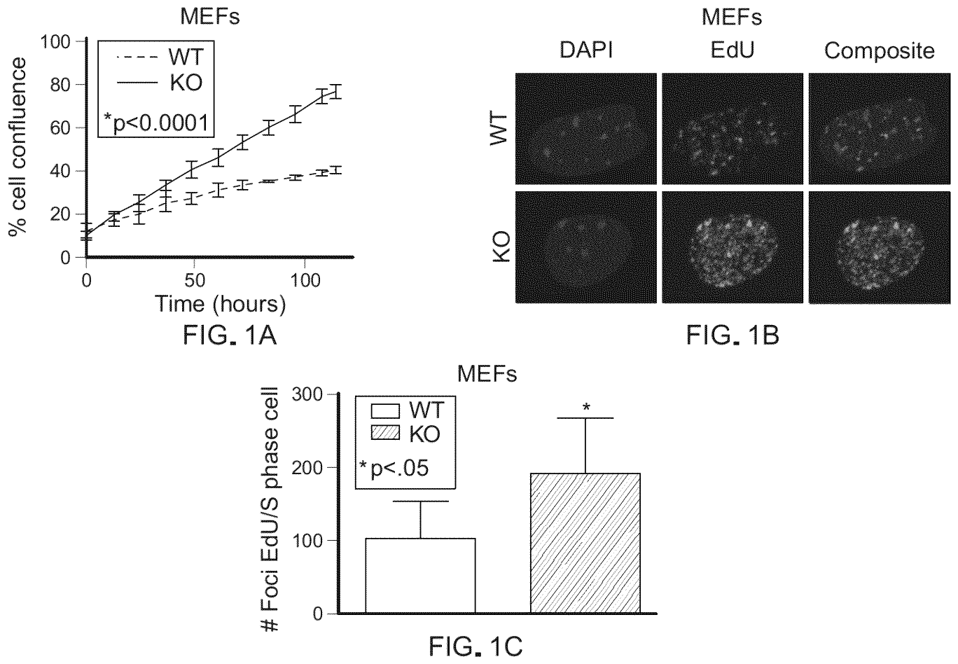

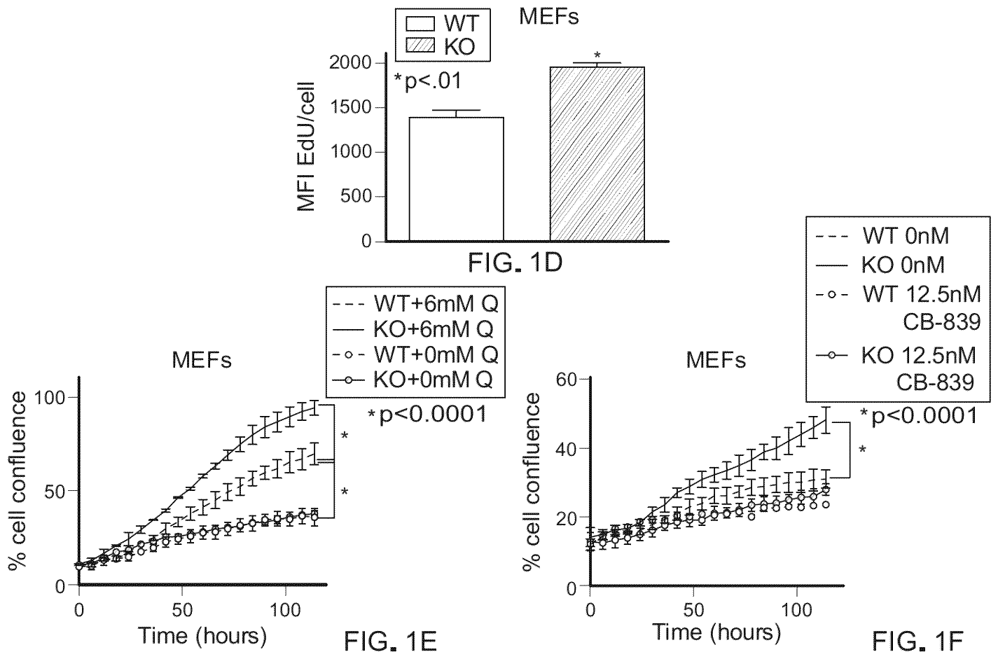

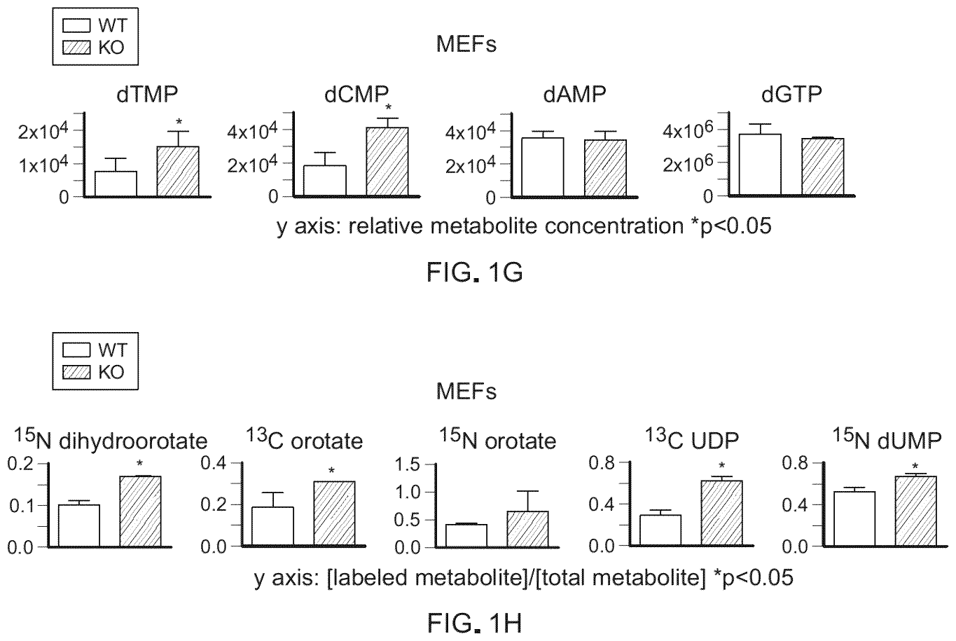

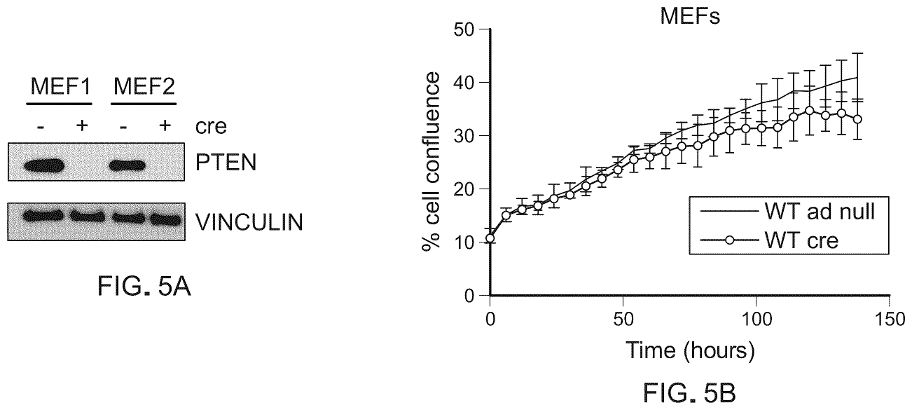

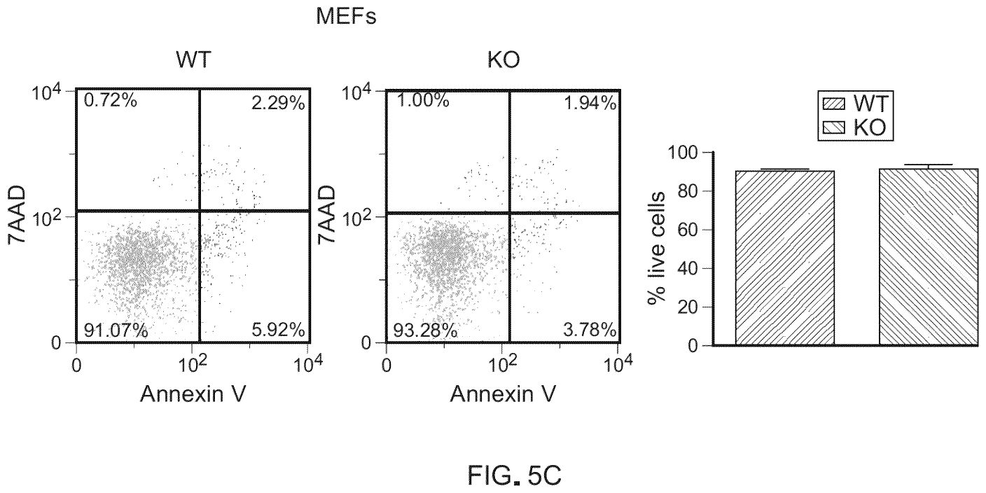

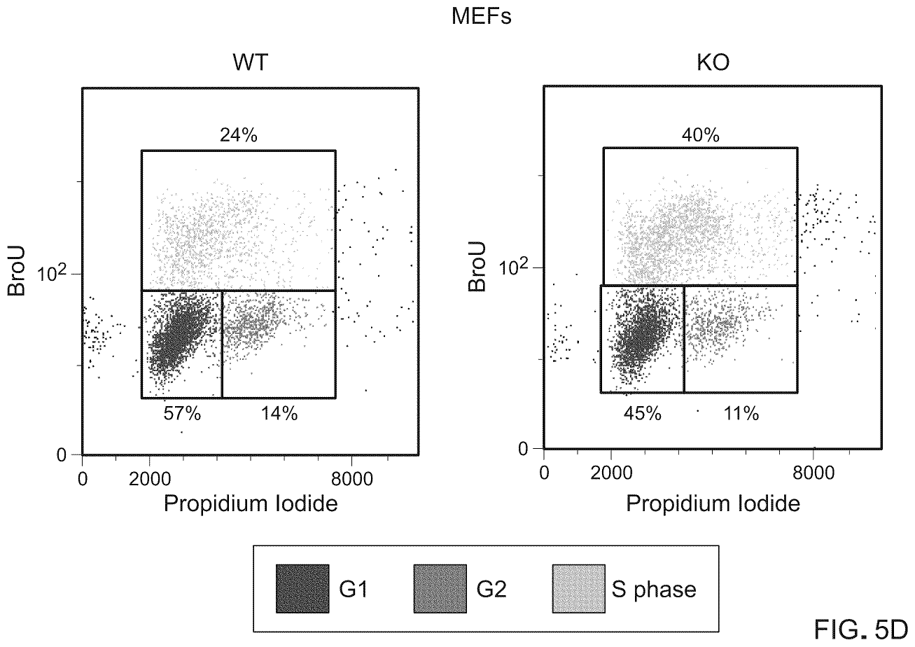

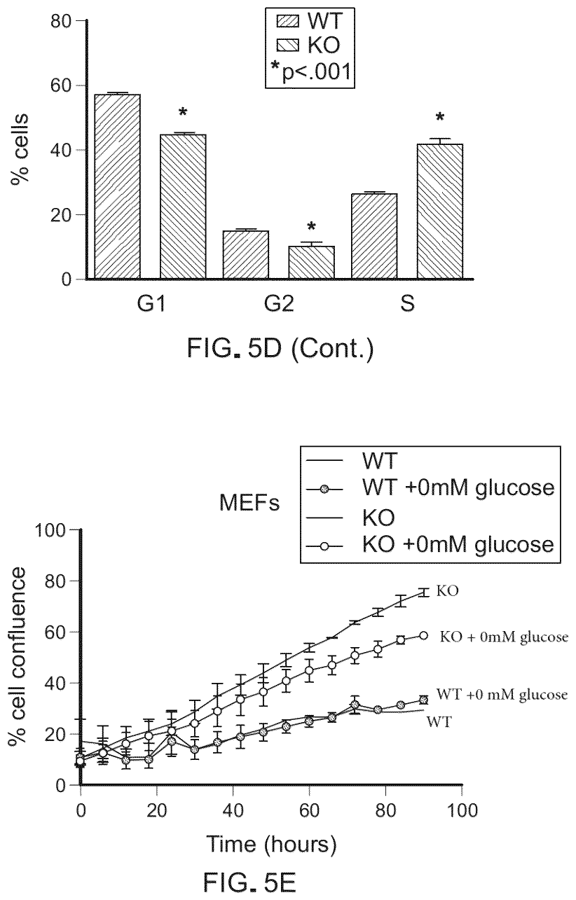

Pten flox/flox (Pten^{-/-}) primary mouse embryonic fibroblasts (MEFs) were generated. Pten-MEFs proliferated at a higher rate than WT MEFs but showed no difference in cell death (FIG. 1A; FIG. 5, A-C). This increased proliferation was associated with an increase in the proportion of cells within S-phase and higher numbers of replication forks per S-phase cell (FIG. 5D; FIG. 1, B-D). Although Pten^{-/-} fibroblasts had elevated glycolytic flux relative to WT fibroblasts, depletion of glucose from the medium was not sufficient to rescue the differences in cell growth (FIG. 5E). Upon testing the potential role of glutamine for explaining the increased growth of Pten−/− cells, it was determined that the growth advantage of Pten^{-/-} MEFs was dependent on glutamine. Specifically, depletion of glutamine or addition of the glutaminase inhibitors CB-839 was sufficient to collapse the growth difference between Pten^{-/-} and WT MEFs (FIG. 1, E-F).

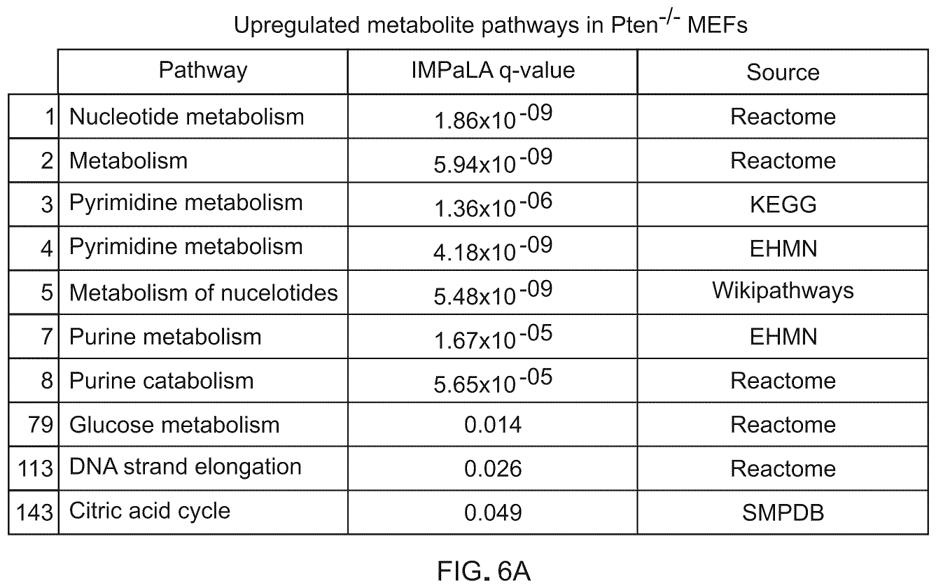

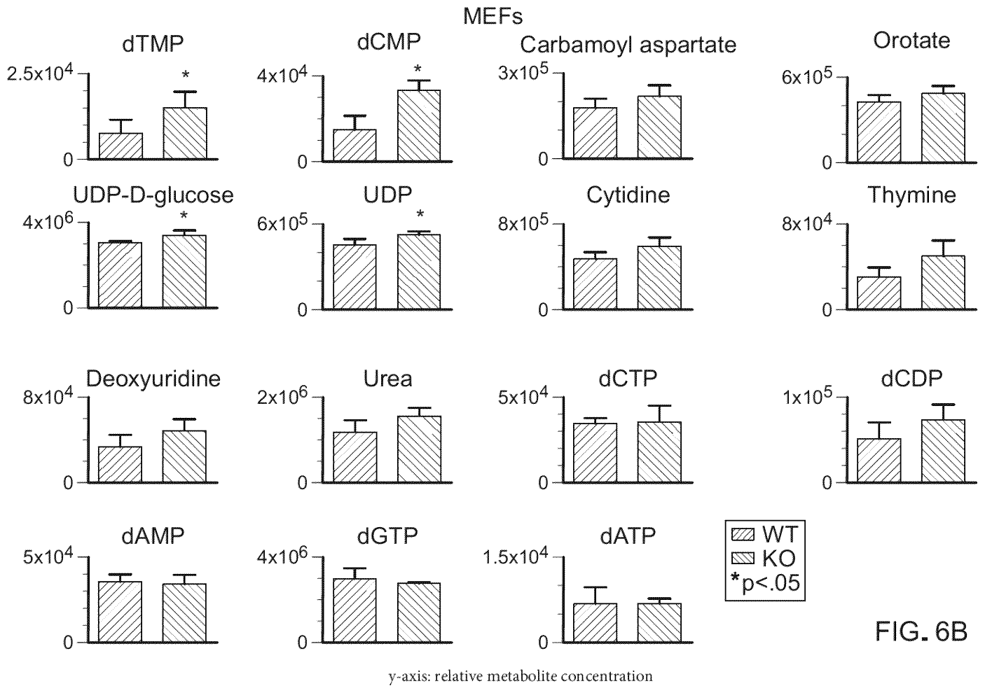

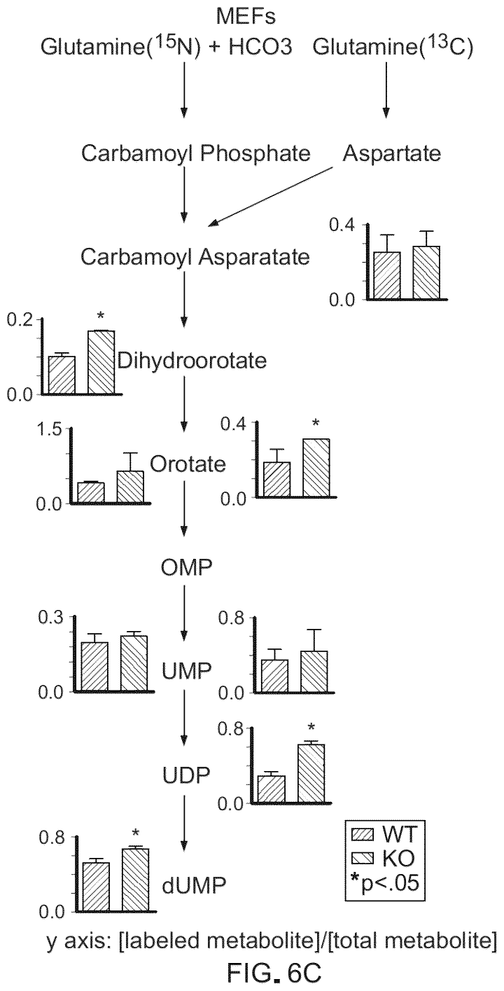

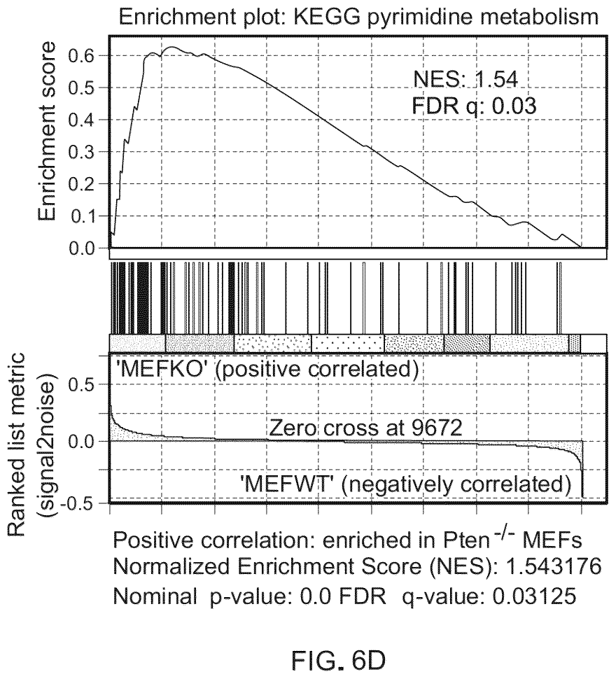

To better understand the relationship between PTEN and glutamine, targeted steady state metabolomic profiling was performed to determine if loss of PTEN triggers abnormal cellular metabolism to increase growth. Unbiased global metabolic assessment of WT and Pten^{-/-} MEFs revealed that seven of the ten most upregulated pathways in Pten^{-/-} MEFs involved nucleotide synthesis and DNA metabolism, including a higher concentration of pyrimidine 2-deoxyribonuceotides in Pten^{-/-} MEFs (FIG. 6, A-B; FIG. 1G). Because glutamine contributes both nitrogen and carbon to pyrimidines, metabolic flux analysis was performed with heavy-isotope 15N or 13C-labeled glutamine, which showed increased synthesis of dihydroorotate, orotate, and other components of the de novo pyrimidine synthesis pathway in Pten/-MEFs relative to WT MEFs (FIG. 1H; FIG. 6C). In addition, the pyrimidine metabolism gene set was upregulated in mRNA from Pten^{-/-} MEFs (FIG. 6D). Nucleotide synthesis is a prerequisite for cellular growth, and Pten/-MEFs appear to channel glutamine for this purpose.

### Example 2: Effect of DHODH Inhibitors on Cell Proliferation

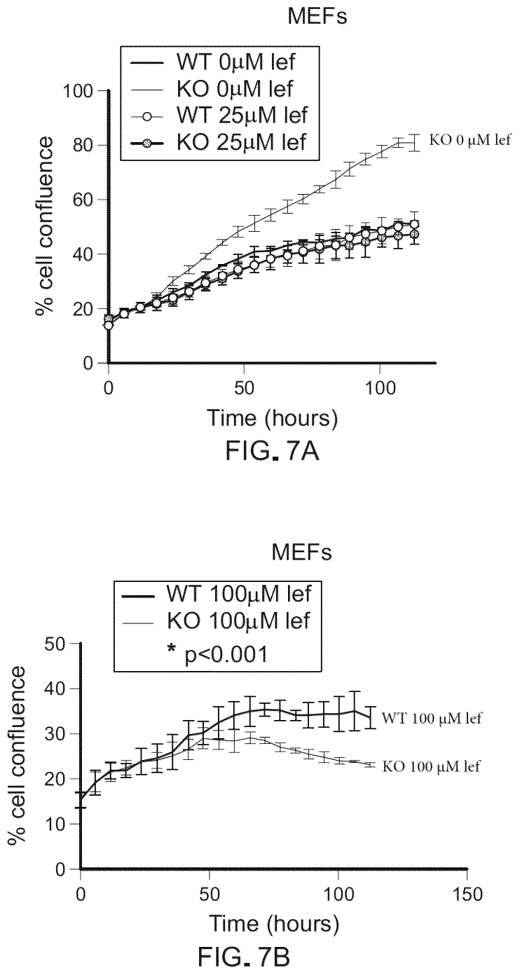

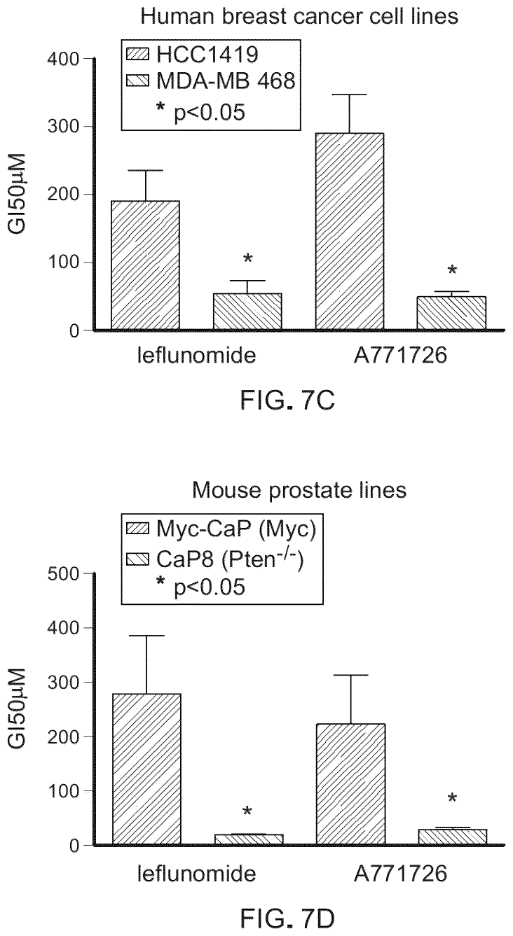

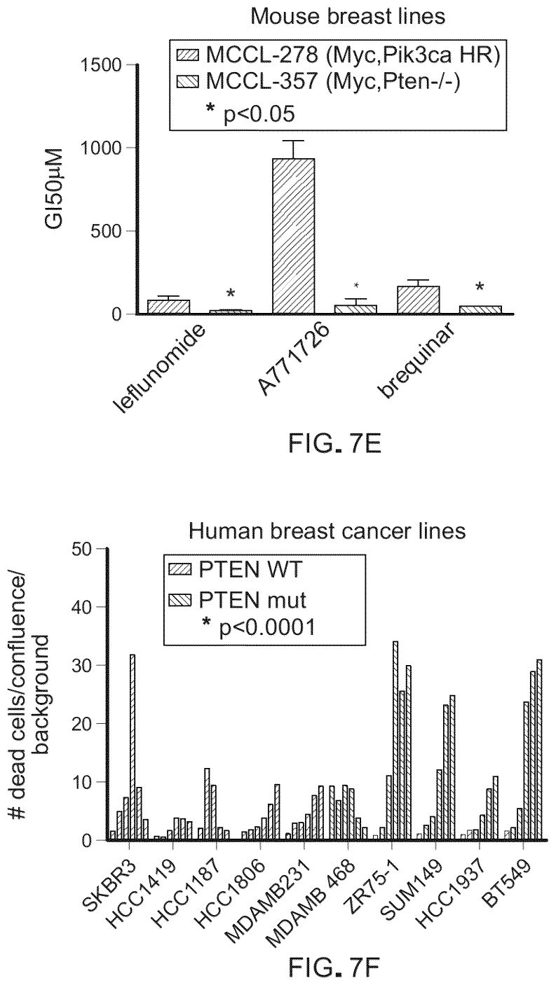

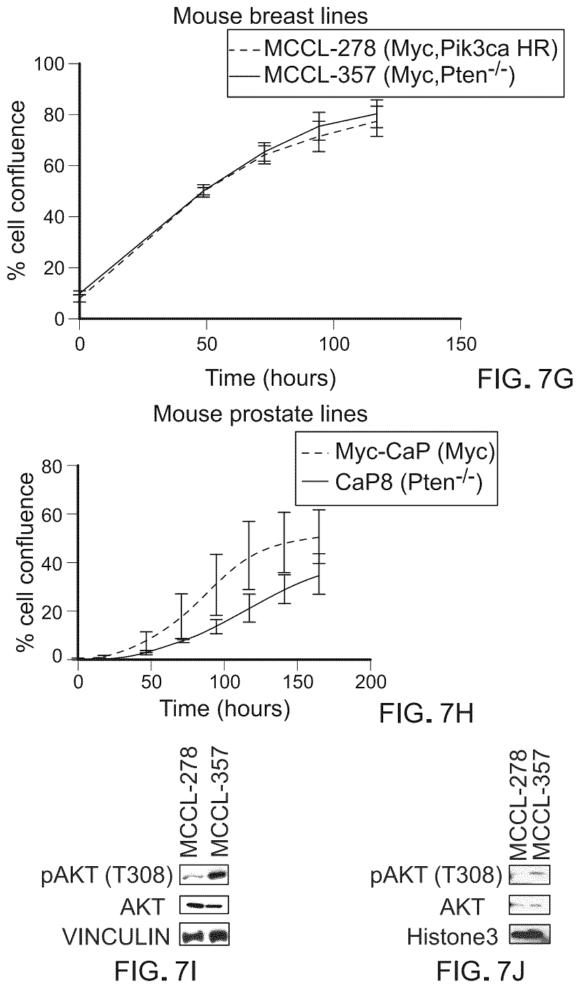

The fourth step of de novo pyrimidine synthesis in mammals is the conversion of dihydroorotate to orotate, catalyzed by dihydroorotate dehydrogenase (DHODH). To determine if orotate contributes to the growth effects observed, the effect of DHODH inhibitors on cell proliferation was examined. Pten-MEFs were about 3-fold more sensitive to a DHODH inhibitor, leflunomide, than WT MEFs were (FIG. 2A; FIG. 7, A-B). Pten^{-/-} MEFs were likewise more sensitive to the active metabolite of leflunomide, A771726, as well as a different DHODH inhibitor, brequinar, indicating that the observed effects were due to inhibition of DHODH and were not limited to a single specific DHODH inhibitor (FIG. 2A).

### Example 3: Relationship Between PTEN Genotype and Sensitivity to DHODH Inhibition

To determine whether PTEN genotype is predictive of sensitivity to DHODH inhibition in cancer cells, multiple human breast, glioblastoma, and prostate cell lines (including SUM149, MDA-MB 468, and BT549) were tested with DHODH inhibitors. Consistently, the GI50 of the PTEN-mutant cells was lower than that of corresponding WT cells (FIG. 2B; FIG. 7C). Mouse cancer lines MCCL-357 (Myc, Pten^{-/-}) and CaP8 (PTEN^{-/-}) were also more sensitive than mouse cancer lines MCCL-278 (Myc, Pik3ca H1047R) and Myc-CaP (Myc) were (FIG. 2C; FIG. 7, D-E). Moreover, Pten^{-/-} MEFs, PTEN-mutant human breast cancer cell lines, and Pten^{-/-} mouse breast lines displayed an increased accumulation of dead cells over time upon treatment with leflunomide (FIG. 2, D-E; FIG. 7F). It is important to note that sensitivity to leflunomide was not associated with the proliferation rates of human breast, mouse breast, or mouse prostate tumor cell lines (FIG. 2F; FIG. 7, G-H). Additionally, it was found that Pten homozygous deletion caused greater AKT phosphorylation than Pik3ca missense mutation did. This was particularly prominent in the nuclear fractions, where AKT may phosphorylate nuclear substrates (FIG. 7, I-J; FIG. 2G).

To independently determine whether DHODH inhibition is detrimental to PTEN-deficient cells, we performed a rescue experiment with orotate, the metabolite directly downstream of DHODH. Increasing concentrations of orotate rescued growth inhibition by leflunomide in a dose-dependent manner (FIG. 2, H-I; FIG. 8A). In addition, siRNA against DHODH preferentially killed PTEN-mutant cells, verifying that DHODH inhibition leads to selective reduction of PTEN-mutant cell growth, despite no difference in DHODH protein level at baseline (FIG. 8, B-C). A771726 also did not affect PI3K signaling (FIG. 8D). Interestingly, treatment with nucleotide analog inhibitors—5-flurouracil or mercaptopurine—did not show a differential sensitivity, demonstrating that Pten^{-/-} MEFs are selectively vulnerable to inhibition of de novo pyrimidine synthesis (FIG. 8, E-F).

Myc activation is known to cause glutamine addiction. CaP8 (PTEN^{-/-}) cells were nearly as sensitive to glutamine deprivation as Myc-CaP cells were, substantiating that a notable level of glutamine dependency is also elicited by PTEN loss (FIG. 8G). Without wishing to be bound by theory, since Myc-CaP cells were resistant to leflunomide, it seems it is not the entry alone of glutamine but its flux into pyrimidines that is important (FIG. 7D). While Myc induction is known to largely direct glutamine to the TCA cycle and phospholipid synthesis, without wishing to be bound by theory, the results of this experiment suggest that Pten loss in MEFs causes glutamine to cascade through the de novo pyrimidine synthesis pathway, creating the point of vulnerability to DHODH inhibition.

### Example 4: Efficacy of DHODH Inhibition in Treating PTEN-Mutant Cancers

To assess the clinical relevance of DHODH inhibitors as targeted cancer therapeutics, patient-derived glioblastomas were grown as 3-dimensional neurospheres and treated with leflunomide. Re-formation of neurospheres was inhibited at lower concentrations of leflunomide in PTEN-deficient samples (FIG. 3A; FIG. 9A) relative to WT samples.

As an additional independent assay, two PTEN-mutant triple-negative breast cancer xenografts were treated with leflunomide, dosing orally as is done clinically. Tumors slowed or regressed upon treatment. Remarkably, even very large tumors (4×10⁷ photons) regressed after only 1 week of treatment, suggesting that DHODH inhibitors can be used for neoadjuvant therapy (FIG. 3, B-C; FIG. 9B). To confirm the specificity of the in vivo effect to PTEN loss, MCCL-357 and MCCL-278 xenografts were treated with leflunomide; as expected, MCCL-357 xenografts had a 4-fold better response than MCCL-278 xenografts did (FIG. 9C).

### Example 5: Mechanism of Action of DHODH Inhibition in Inducing PTEN-Mutant Cell Death

It was unclear why DHODH inhibitors were selectively cytotoxic to Pten^{-/-} cells. Although DHODH inhibitors were known to be generally cytostatic (as inhibitors of pyrimidine synthesis), this effect would have an equal impact on both Pten^{-/-} and WT cells. Consistent with prior reports, Pten^{-/-} MEFs had a higher baseline level of gamma-H2AX, an indicator of DNA damage (FIG. 4A). Leflunomide (or A771726) augmented DNA damage to a significantly greater degree in PTEN-deficient cells; this damage co-localized with replication forks labeled with EdU (FIG. 4, B-C; FIG. 10, A-C). The greater number of replication forks in Pten^{-/-} MEFs remained intact after 24 h of treatment with leflunomide, showing that the cells continued to replicate despite the presence of DNA damage, which suggested that they were not responding with appropriate S-phase checkpoints to the DNA damage (FIG. 1B, 4D; FIG. 10, D-E).

Depletion of nucleotide pools normally activates the ATR checkpoint at replication forks in S-phase cells. ATR checkpoint activation at stalled forks requires two signals, one through single-strand DNA binding protein (RPA) interaction with single-strand DNA to recruit the ATRIP-ATR complex, and a second signal through TOPBP1 interaction with the ATR activation domain. Deletion of PTEN in cells causes poor ATR checkpoint activation, which is due to AKT phosphorylation of TOPBP1 on serine 1159 and CHK1 on serine 280. To further investigate the response to DNA damage occurring at Pten/-forks, the interaction of RPA and gamma-H2AX was examined by flow cytometry. An increase in RPA signal was first achieved regardless of PTEN genotype in the presence of A771726, followed by a shift toward both RPA and gamma-H2AX-positive cells in Pten^{-/-} MCCL-357 but not in Pten WT MCCL-278 cells (FIG. 4E). Furthermore, gamma-H2AX appeared almost exclusively in RPA-positive MCCL-357 cells treated with A771726 (FIG. 10F). A771726 also triggered ATR phosphorylation of CHK1 at serine 345 in Pten WT, but to a much lesser extent in Pten^{-/-} cells (FIG. 4F; FIG. 10G). These data indicate that Pten^{-/-} cells are incapable of generating an appropriate activation of the ATR-CHK1 checkpoint at replication forks. Activation of CHK1 in MCCL-278 cells declined as RPA declined, suggesting that Pten WT cells eventually recovered from DHODH inhibition, while Pten^{-/-} cells instead accumulated damage at 18 h (FIG. 4F). By 48 h, this genomic stress manifested in a greater number of chromosome gaps, breaks, and multiradial formations in MCCL-357 cells treated with A771726 compared to MCCL-278 cells (FIG. 4, G-H; FIG. 10, H-I). These findings are consistent with the sensitivity to hydroxyurea that occurs in the setting of an ATR inhibitor.

### Example 6: Proposed Phase II Clinical Trial of DHODH Inhibition Vs. Physician/Patient's Choice of Chemotherapy Regimen in Previously Treated Triple-Negative Breast Cancer

Patients are women ≥18 with metastatic measurable or evaluable triple-negative breast cancer who have previously been treated with 1-3 chemotherapy regimens for metastatic disease. Specifically, patients are required to have an ECOG performance of 0-2 and a histologically confirmed pre-trial biopsy of an accessible site of metastatic disease that shows ER (≤1%), PR (≤1%), and HER2 negative (either by immunohistochemistry score of 0, or FISH or ISH<2.0. Patients are additionally required to have been last treated with oral or IV chemotherapy, small molecule inhibitors, biologic agents, surgery, or radiation 4 or more weeks prior to starting the trial. Patients with a history of previously treated brain metastases are required to have been last treated with definitive surgery, gamma knife/whole brain radiation, or steroids 4 or more weeks prior to starting the trial. Patients are required to have lab parameters including white blood cell count and lymphocytes within an institution-defined normal range; hemoglobin ≥9 g/dl; platelets ≥100,000/μl; liver function (as assessed by ALT and AST)≤1.5 times the upper limit of an institution-defined normal range or (for patients with liver metastases) ALT and AST≤2 times the upper limit of the normal range; and creatinine≤1.5 times the upper limit of an institution-defined normal range. Patients are permitted to take denosumab or zoledronic. All patients that do not have a documented negative TB test within 1 year of study entry will be required to undergo a TB skin test. Patients of childbearing age are required to have a negative urine or serum pregnancy test prior to starting the trial, and are further required to agree to use birth control (IUD, diaphragm, condoms, tubal ligation, or (for their male partners) vasectomy) for the duration of the trial and for 8 weeks after the conclusion of the trial. Patients are not permitted to use cholestyramine, rifampin, or itraconazole for the duration of the trial. Patients are not permitted to use or participate in any other standard or investigational oral or IV chemotherapy or radiation regimens for the duration of the trial.

Patients are required to have no history of hypersensitivity to teriflunomide (the active metabolite of leflunomide); no history of hepatitis B, hepatitis C, human immunodeficiency virus (HIV), or tuberculosis; no prior history of non-breast other cancers (except cervical carcinoma in situ and basal cell cancers); no history of interstitial lung disease; no untreated brain metastases; no carcinomatous meningitis; no pre-existing acute or chronic liver disease; no uncontrolled serious medical or psychiatric condition that would potentially interfere with informed consent or compliance with required study endpoints or medications.

Patients are treated with a DHODH inhibitor, leflunomide or teriflunomide, or their physician's choice of chemotherapy regimen for 12 weeks, at which time outcomes will be assessed. Patients who are found to be responding to their assigned therapeutic regimen (i.e., DHODH inhibition or chemotherapy) at 12 weeks will be kept on the regimen until cancer progression, the occurrence of dose-limiting side effects, or voluntary withdrawal from the trial. Patients on either therapeutic regimen (i.e., DHODH inhibition or chemotherapy) who experience cancer progression are allowed to switch to the other regimen at their or their physician's discretion.

This is a Phase II randomized trial with 2:1 treatment assignment (2 leflunomide/teriflunomide: 1 standard chemotherapy regimen selected by the patient or their physician), with an early stopping rule for the leflunomide/teriflunomide arm. Patients randomized to the leflunomide/teriflunomide arm will receive 14 mg oral teriflunomide per day. Treatment will continue daily until progression of disease, adverse side effects, or voluntary withdrawal from trial. Patients randomized to the standard chemotherapy arm will be treated with their or their physician's choice of chemotherapy regimen.

The required baseline and subsequent tests are outlined in the table below:

TABLE 2
Required baseline and subsequent tests

| | | Weekly, | | | |
|---|---|---|---|---|---|
| | | beginning | Every | Every | |
| | | with day | 3 or 4 | 9-12 | |
| Test | Baseline^{§} | 1, cycle 1 | weeks | weeks^{¶} | Off Trial |
| History/PE/ECOG | X | | X | | |
| CBCD | X | X | X | | X |
| CMP | X | | X | | X |
| Hepatic Function | X | X | X | | X |
| Serum Tumor Marker | X^{∞} | | X | | X |
| TB test | X | | | | |
| Urine or serum | X | | | | |
| pregnancy test | | | | | |
| PET/CT or CT scan of | X | | | X | |
| chest, abdomen and | | | | | |
| pelvis and bone scan^{#} | | | | | |
| [symbol] | X | | X | | X |
| Tumor Biopsy | X⁺ | | | | X^{≈} |

Abbreviations:
physical exam (PE); Eastern Cooperative Oncology Group Performance Status (ECOG PS); complete blood count and differential (CBCD); complete metabolic panel (CMP); health-related quality of life (HRQOL).
^{§}Within 4 weeks prior to starting day 1, cycle 1
^{¶}Depending on standard chemotherapy a cycle is usually 3 or 4 weeks.
^{∞}At the baseline blood collection measure tumor markers. If one is elevated then use the same one at every time point.
^{#}Use the same imaging modality at every time point.
⁺Baseline biopsy is mandatory.
^{≈}Optional biopsy within 4 weeks after stopping study drug. [symbol]
-Related Quality of Life (HRQOL) will be measured using the NCI

Patient-Reported Outcomes Measurement Information System (PROMIS®) (see, e.g., www.nihpromis.org). NCI has developed PROMIS to standardize instruments across the domains of health-related quality of life as using patient-reported outcomes to rate side effects of treatment may represent a more accurate method of capturing patient experiences. The Global Health Scale (see Appendix) is a 10-question HRQOL assessment tool that elicits information on patients' perceived overall HRQOL. A physical health score and mental health score are derived from the Global Health Scale. The scores are calibrated on a T-score metric normed with a general population sample with a mean of 50 and a standard deviation of 10. Higher scores reflect better HRQOL. In addition, specific scales using PROMIS® short forms including depression, anxiety and social isolation (see Appendix) will also be administered. PROMIS® has been used with cancer patients during chemotherapy.

For patients in the leflunomide/teriflunomide arm, the following dose delays and modifications will apply:

First occurrence: if grade 4 hematologic, or grade ≥3 or higher non-hematologic occurs hold dose of leflunomide or teriflunomide up to 2 weeks until it resolves ≤grade 1. Restart at same dose.

Second occurrence: if grade 4 hematologic, or grade ≥3 or higher non-hematologic occurs hold dose of leflunomide or teriflunomide up to 2 weeks until it resolves ≤grade 1. Restart at 50% dose reduction,

Third occurrence: if grade 4 hematologic, or grade ≥3 or higher non-hematologic occurs, remove from trial.

The definitions used above are standard definitions known in the art; see, e.g., the NCI Common Terminology Criteria for Adverse Events (CTC-AE), version 4.

For patients in the randomized chemotherapy arm, the same definitions of first, second, and third occurrences given above for patients in the leflunomide/teriflunomide arm will be used; however, dose reductions will be according to the physician's judgement.

Metastatic sites will be characterized at the pre-treatment baseline imaging as measurable or non-measurable as per RECIST 1.1. Sites must be accurately measured in at least one dimension (longest diameter in the plane of measurement is to be recorded) with a minimum size of 10 mm by CT scan (irrespective of scanner type) and MRI (no less than double the slice thickness and a minimum of 10 mm), 10 mm caliper measurement by clinical exam (when superficial), or 20 mm by chest X-ray (if clearly defined and surrounded by aerated lung). Non-measurable metastatic sites include, e.g., leptomeningeal disease, ascites, pleural or pericardial effusion, inflammatory breast disease, lymphangitic involvement of skin or lung, and abdominal masses/abdominal organomegaly identified by physical exam that is not measurable by reproducible imaging techniques.

Lymph nodes are categorized/defined as normal: short axis <10 mm, measurable (target): short axis ≥15 mm, and non-measurable: short axis 10 mm to <15 mm. Target nodes measured in the short axis (perpendicular to longest diameter) are more reproducible and predictive of malignancy. Short axes of target nodes will be added to the sum of longest diameters.

Lytic bone lesions with an identifiable soft tissue component, evaluated by CT or MRI, can be considered measurable lesions if the soft tissue component otherwise meets the definition of measurability described above. Blastic bone lesions are non-measurable.

Cystic lesions that meet radiographic criteria for simple cysts will not be considered as malignant lesions (neither measurable nor non-measurable). Radiographically indeterminate, complex “cystic” lesions will be considered non-measurable lesions. “Cystic lesions” thought to be cystic metastases can be considered measurable lesions, if they meet the definition of measurability described above. However, if non-cystic lesions are present in the same patient, these will preferably selected for assessment.

All lesions up to a maximum of five lesions total (and a maximum of two lesions per organ) representative of all involved organs will be identified as target lesions. It may be the case that, on occasion, the largest lesion does not lend itself to reproducible measurement, in which circumstance the next largest lesion that can be measured reproducibly will be selected. Tumor lesions situated in a previously irradiated area, or in an area subjected to other loco-regional therapy, will generally not be considered measurable unless there is demonstrated progression in the lesion.

“Complete response (CR)” to a treatment is defined as the disappearance of all target lesions (i.e., any pathological lymph nodes (whether target or non-target) must have reduction in short axis to <10 mm (the sum may not be “0” if there are target nodes) and the disappearance of all non-target lesions and normalization of tumor marker level (i.e., all lymph nodes must be non-pathological in size (<10 mm short axis).

“Partial response (PR)” to a treatment is defined as a ≥30% decrease in the sum of diameters of the target lesions, taking as reference the baseline sum of diameters of all lesions.

“Stable disease (SD)” to a treatment is neither sufficient shrinkage to qualify for a PR nor sufficient increase to qualify for progressive disease (PD).

“PD” to a treatment is defined as a ≥20% increase in the smallest sum of the diameters on the study (or taking as a reference the baseline if that is the smallest on study), wherein the increase is at least 5 mm. The appearance of one or more new lesions is also considered progressive disease.

“Unequivocal progression” of existing non-target lesions is defined as an overall level of substantial worsening in non-target disease such that, even in presence of SD or PR in target disease, the overall tumor burden has increased sufficiently to merit discontinuation of therapy. Alternately, in the absence of measurable disease, “unequivocal progression” is defined as a change in non-measurable disease comparable in magnitude to the increase that would be required to declare PD for measurable disease. Examples of “unequivocal progression” include, e.g., an increase in a pleural effusion from ‘trace’ to ‘large’ or an increase in lymphangitic disease from localized to widespread.

All target lesions (nodal and non-nodal) recorded at baseline will have their actual measurements recorded at each subsequent evaluation, even when very small (e.g. 2 mm). However, if target lesions or lymph nodes become so faint on CT scan that the radiologist is not able to assign an exact measure, a default value of 5 mm will be assigned.

When non-nodal lesions ‘fragment’, the longest diameters of the fragmented portions will be added together to calculate the target lesion sum. Similarly, as lesions coalesce, a plane between them may be maintained that would aid in obtaining maximal diameter measurements of each individual lesion. If the lesions have truly coalesced such that they are no longer separable, the vector of the longest diameter in this instance should be the maximal longest diameter for the ‘coalesced lesion.’

Finding of a new lesion should be unequivocal, i.e., not attributable to differences in scanning technique, change in imaging modality, or findings thought to represent something other than a tumor. When unclear, one or more subsequent time points will be evaluated when possible. Lesions seen in an anatomical region that was not imaged at baseline will be considered new lesions.

When no imaging/measurement is done at all at a particular time point, the patient is considered not evaluable (NE) at that time point. If only a subset of lesion measurements are made at an assessment, the case will also generally be considered NE at that time point, unless evidence indicates that the contribution of the individual missing lesion(s) would not change the assigned time point response.

If a lesion disappears (or appears to disappear—e.g., if a lesion is beyond the resolving power of the imaging modality employed) and reappears (or appears to reappear) at a subsequent time point, it will continue to be measured and will simply be added into the sum in determining the patient's overall response.

Time to progression is considered the primary endpoint. Response (CR, PR, SD, or PD) is considered the secondary endpoint (confirmation is not required).

A total of 105 patients will be randomized in a 2:1 allocation (70 patients to leflunomide or teriflunomide and 35 patients to physician/patient's choice of chemotherapy). This sample size will allow detection of a difference between a median progression-free survival (PFS) of 5 months for the leflunomide/teriflunomide arm and a median PFS of 2.5 months for the physician/patient's choice of chemotherapy arm with power 0.90 for a two-sided test at the 0.05 level. A drop-out rate of 10% is assumed and hence the total sample size will be increased to 116 patients.

PFS is defined as the time from the date of randomization to confirmed disease progression or death from any cause, whichever comes first. Subjects who withdraw from the study or are considered lost to follow-up without prior documentation of disease progression will be censored on the date of the last disease assessment. Randomization will be stratified by PTEN status (present vs. absent). The prevalence of PTEN is about 50% in metastatic triple-negative breast cancer. Hence, approximately 58 patients will have PTEN present and 58 patients will have PTEN absent. Each PTEN stratum will therefore randomize 39 patients to receive leflunomide and 19 patients to receive standard treatment. An intent to treat (ITT) analysis to compare PFS for the two treatment arms will be by a stratified log-rank test and also by separate analyses by PTEN presence or absence. The possibility of an interaction effect between treatment and PTEN status will also be assessed. A Cox regression model for PFS with only treatment (leflunomide vs. standard of care) and PTEN status (present vs. absent) in the model will be tested against a model containing these variables plus an interaction term, by using a likelihood ratio statistic. This procedure will determine if there is a differential treatment effect based on PTEN status.

There will be a stopping rule based on lack of a sufficient overall response rate (RR) for each of the leflunomide/teriflunomide arms within the two PTEN strata. This requires that a minimum of 35 patients be the target sample size for leflunomide arms. A Simon optimal two stage design for each leflunomide group will be used. If ≤2 responses out of the first 18 patients are observed in a leflunomide arm (PTEN present or absent stratum), that arm will not accrue any further patients. If there are more than 2 responses, an additional 17 patients will be accrued. This design has a type I error probability of 0.05 and a type II error probability of 0.10 for a test with a null hypothesis that the true response rate <0.10 versus an alternative hypothesis that the true response rate >0.30.

There will also be a stopping rule for safety/toxicity. If 2 or more of the first 10 patients in a leflunomide/teriflunomide arm (irrespective of PTEN status) experience grade 3 or higher toxicities, that arm will be closed. The probability of observing such events in fewer than 2 of 10 patients if the true but unknown toxicity rate is 30% is 0.20; if the true but unknown rate for such toxicities is 50%, the probability of observing such events in fewer than 2 patients is 0.02.

### Methods

### Immunoblotting:

Samples were lysed in 2× Laemelli sample buffer with mercaptoethanol and were boiled before separation by SDS-PAGE on Tris-Glycine gels (Invitrogen EC60352). Wet-transfer to PVDF (Fisher ipvh00010) was followed by blocking for 1 hour in 10% nonfat milk (Fisher M-0841) in TBST. Membranes were incubated in primary antibody overnight at 4° C., and washed with TBST prior to addition of secondary antibody (Fisher 31432, 31460) for 1 hour at room temperature. Blots were developed using ECL (Fisher 34080) and autoradiography film (Denville E3018). Primary antibodies: PTEN 6H2.1 (Millipore 04-035), DHODH (Proteintech 14877-1-AP), vinculin (Sigma), pChk1 (Cell Signaling 2341), and Chk1 G-4 (Santa Cruz sc-8408).

### Cell Culture:

MEFs and mouse breast tumor lines: DMEM (Corning mt10013cv) supplemented with 10% FBS (Atlanta Biologicals), 1% pen/strep (Fisher 30002ci) and 2 mM L-glutamine (total 6 mM) (Fisher MT25005CI). MDA-MB 468, MDA-MB 231, Myc-CaP, and U87: DMEM supplemented with 10% FBS and 1% pen/strep. HCC1419, HCC1187, HCC 1937, HCC 1806, BT549, ZR75-1, PC3, LNCAP, DBTRG: RPMI (Fisher 10040cv) supplemented with 10% FBS and 1% pen/strep. CaP8 cells: DMEM with 10% FBS, 1% pen/strep, and 5 μg/mL insulin (Sigma 19278). Neurospheres: stem cell media with 10 μg/mL FGF (R&D Systems 233-FB-025), 20 μg/mL EGF (Peprotech AF-100-15) and heparin. All cells were cultured in a 37° C. incubator with humidity and 5% CO₂. Cell lines were obtained from ATCC, with the exception of MEFs, MCCL-278, and MCCL-357 which were generated from mice. Cell lines were clear of mycoplasma as determined by the Lonza kit (LT07-418) within 6 months of their use.

### Mouse Embryonic Fibroblasts (MEFs):

Embryos were harvested from pregnant B6.129S4 Pten flox/flox mice (Jackson Laboratory) 14 days after mating. Head, limbs, liver, and other highly vascularized regions of the embryo were removed. The remaining trunk was minced using a scalpel in 0.25% trypsin, and resuspended using a 5 mL pipet. After 10 min incubation at 37° C. and 5% CO₂, cells were further resuspended with a 1 mL pipet to single-cell suspension. Cells were resuspended in fresh media before plating onto 10 cm dishes. Cells were treated with an adenovirus with or without cre recombinase, and were studied within 2-5 passages post infection.

### Proliferation Assay:

1500 cells per well (mouse cells) or 3000 cells per well (human cells) were plated in 96-well plates (Corning 720089). Growth rates were determined using the phase-confluency readings on an IncuCyte ZOOM (Essen Biosciences) on live cells over time.

### Metabolite Labeling:

Media without added glutamine was supplemented with 13C glutamine (fully labeled) or 15N glutamine (amide labeled) (Cambridge Isotope Labs). Cells were plated in 10 cm dishes and grown in normal media. 1 h prior to metabolite extraction, media was aspirated and replaced with heavy isotope-labeled media.

### Metabolic Extraction:

Metabolites were extracted in methanol. Specifically, media was aspirated from plates, and 2.5 mL 80% methanol (kept at −80° C.) was added. Plates were incubated at −80° C. for 20 minutes, after which cells were scraped into tubes and centrifuged to pellet insoluble cellular material. The soluble supernatant was saved. Two more extractions on the insoluble pellet were performed with 500 μL 80% methanol, and all extractions were pooled. Extractions were dried in a speed-vac and frozen at −80° C. until analysis. All steps of the extraction were kept cold on dry ice.

### Targeted Mass Spectrometry:

Mass spectrometry was performed by the core facility at Beth Israel Deaconess Medical Center. Samples were re-suspended using 20 μL HPLC grade water for mass spectrometry. 5-7 μL were injected and analyzed using a hybrid 5500 QTRAP triple quadrupole mass spectrometer (AB/SCIEX) coupled to a Prominence UFLC HPLC system (Shimadzu) via selected reaction monitoring (SRM) of a total of 259 endogenous water soluble metabolites for steady-state analyses of samples. Some metabolites were targeted in both positive and negative ion mode for a total of 294 SRM transitions using positive/negative ion polarity switching. ESI voltage was +4900V in positive ion mode and −4500V in negative ion mode. The dwell time was 3 ms per SRM transition and the total cycle time was 1.55 sec. Approximately 10-14 data points were acquired per detected metabolite. Samples were delivered to the mass spectrometer via hydrophilic interaction chromatography (HILIC) using a 4.6 mm i.d×10 cm Amide XBridge column (Waters) at 400 μL/min. Gradients were run starting from 85% buffer B (HPLC grade acetonitrile) to 42% B from 0-5 minutes; 42% B to 0% B from 5-16 minutes; 0% B was held from 16-24 minutes; 0% B to 85% B from 24-25 minutes; 85% B was held for 7 minutes to re-equilibrate the column. Buffer A was comprised of 20 mM ammonium hydroxide/20 mM ammonium acetate (pH=9.0) in 95:5 water: acetonitrile. Peak areas from the total ion current for each metabolite SRM transition were integrated using MultiQuant v2.1 software (AB/SCIEX). ˜150 SRM transitions were set up for 13C glutamine and 15N glutamine labeled metabolites in addition to unlabeled metabolites. Integrated Molecular Pathway Analysis (IMPaLA) was used to analyze metabolic pathways. For cell-labeling experiments, the concentration of isotope-labeled metabolite=[labeled metabolite amount]/[total metabolite amount] for each metabolite.

### Gene Set Enrichment Analysis:

Microarray data from Pten WT and KO MEFs (4 each) were analyzed using the GSEA program by the Broad Institute.

### Cell Cycle Analysis:

The FlowCellect™ Bivariate Cell Cycle Kit (Millipore FCCH025102) was used according to the instructions provided in the kit. Fluorescence was measured on a Guava® flow cytometer. BrdU was pulsed for 18 h.

### Cell Death:

The FlowCellect™ Annexin Red Kit (Millipore FCCH100108) was used according to the instructions provided in the kit. Fluorescence was measured on a Guava® flow cytometer.

### Drug Response Assays:

Cells were plated in 96-well plates at a density of 1500 or 3000 cells per well. Leflunomide (Sigma PHR1378-1G), A771726 (Sigma SML0936), mercaptopurine (Sigma 852678), brequinar (Sigma SML0113), 5-fluorouracil (Millipore 343922), and CB-839 (MedChemexpress HY-12248) were dissolved in DMSO. Sensitivity was determined by a dose-response titration for each cell line, with an equivalent amount of DMSO in each well. For cell death assays, DRAQ7™ (Cell Signaling 7406S) was added to the media at a 1:200 dilution and red fluorescence was measured in addition to phase in live-cell imaging to measure accumulation of dead cells. An IncuCyte ZOOM was used.

### Gamma-H2AX Measurement:

The FlowCellect™ Cell Cycle Checkpoint H2A.X DNA Damage Kit (Millipore FCCH12542) was used according to the instructions provided in the kit. Briefly, cells were fixed and permeabilized, followed by staining with an anti-phospho-H2A.X antibody and propidium iodide. For co-staining with RPA, an additional step was performed during which cells were incubated with an RPA antibody (Abcam ab79398) for 1 h and secondary antibody for 1 h. (Propidium iodide was not used in this setting.) Fluorescence was measured on a Guava® flow cytometer.

### Edu Detection:

The EdU Cell Proliferation Kit (Millipore 17-10525) was used according to the instructions provided in the kit. Briefly, cells were fixed and permeabilized following a 45 min EdU pulse. Fluorescence was measured on a Guava® flow cytometer or by immunofluorescence.

### Immunofluorescence:

Cells were plated on cover slips in media. For detecting replication forks: following a 45 min EdU pulse, cover slip-attached cells were fixed and permeabilized, and stained with an EdU-binding azide dye. For detecting gamma-H2AX: cells were incubated with primary antibody (Upstate Cell Signaling) overnight at 4° C. and with secondary antibody for 2 h at room temperature. Images were taken using a Zeiss LSM880 Airyscan confocal microscope at 63X, and foci number and colocalization was quantified with ImageJ.

### Karyotyping:

Chromosomal analysis was done as follows: Mouse PTEN−/− and PTEN WT cells were sub-cultured and the drug was added at the indicated concentrations 24 h after sub-culturing. The cells were processed for metaphases preparations by standard protocols after 48 h and 72 h of drug exposure with the addition of colcemid for the last 2 h. A total of 100 metaphases were analyzed from replicate experiments to identify chromatid- and chromosome-type aberrations such as chromatid and chromosome breaks, multi-radial chromosomes, extensive breakage resulting in pulverization. Chromatid and chromosome breaks were considered as a single break, multi-radial chromosomes were considered as 3 breaks in assessing the frequency of abnormal metaphases and chromosome breaks. However, extensive breakage resulting in pulverization in rare metaphases was not considered in calculating the frequency of breaks. Experiments were repeated twice.

### Orotate Rescue:

Orotate (Sigma O2750) was dissolved in DMSO.

### RNA Interference:

siRNA for DHODH was obtained from Qiagen. Cells were transfected using lipofectamine (Invitrogen 11668-019) and knockdown was confirmed at 48 h. Scrambled siRNA was used as a control.

### Xenografts:

6-week old nu/nu mice (Jackson Laboratory, 20-25 g weight each) were engrafted orthotopically with either 5 million SUM149, 5 million MDAMB 468-luciferase, 1 million MCCL-357, or 0.75 million MCCL-278 cells. Mice were treated by oral gavage with 100 mg/kg leflunomide or vehicle control (1% carboxymethylcelluose in water). Luminescence was measured on days 0, 7, 14, and 21, quantified as photons/second/cm²/steradian, and normalized to baseline. Mice were treated orally as is done clinically; leflunomide binds tightly to serum proteins and has a long half-life (˜2 weeks), precluding daily treatments for the duration of the experiment.

### Neurosphere Sensitivity Assay:

Neurospheres were disrupted by manual pipetting until single cell suspension was achieved, and 10,000 cells/well were plated in low-attachment 6-well plates (Fisher 3471). After 5 days, neurosphere formation was counted; sphere-forming ability is an indicator of tumorigenicity.

### Statistical Analysis:

ANOVA or Student's t-tests were used to test means between groups. Correction for multiple comparisons was added where needed. Analyses were performed using GraphPad Prism 6.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Dang CV. Links between metabolism and cancer. Genes Dev. 2012; 26:877-90.
2. Shim H, Chun Y S, Lewis B C, Dang C V. A unique glucose-dependent apoptotic pathway induced by c-Myc. Proc Natl Acad Sci USA 1998; 95:1511-6.
3. Elstrom R L, Bauer D E, Buzzai M, Karnauskas R, Harris M H, Plas D R, et al. Akt stimulates aerobic glycolysis in cancer cells. Cancer Res. 2004; 64:3892-9.
4. Wise D R, DeBerardinis R J, Mancuso A, Sayed N, Zhang X-Y, Pfeiffer H K, et al. Myc regulates a transcriptional program that stimulates mitochondrial glutaminolysis and leads to glutamine addiction. Proc Natl Acad Sci USA 2008; 105:18782-7.
5. Ben-Sahra I, Howell J J, Asara J M, Manning B D. Stimulation of de novo pyrimidine synthesis by growth signaling through mTOR and S6K1. Science 2013; 339:1323-8.
6. Li J, Yen C, Liaw D, Podsypanina K, Bose S, Wang S I, et al. PTEN, a putative protein tyrosine phosphatase gene mutated in human brain, breast, and prostate cancer. Science 1997; 275:1943-7.
7. Steck P A, Pershouse M A, Jasser S A, Yung WKA, Lin H, Ligon A H, et al.

Identification of a candidate tumour suppressor gene, MMAC1, at chromosome 10q23.3 that is mutated in multiple advanced cancers. Nat Genet. 1997; 15:356-62.
8. Liaw D, Marsh D J, Li J, Dahia PLM, Wang S I, Zheng Z, et al. Germline mutations of the PTEN gene in Cowden disease, an inherited breast and thyroid cancer syndrome. Nat Genet. 1997; 16:64-67.
9. Maehama T, Dixon J E. The tumor suppressor, PTEN/MMAC1, dephosphorylates the lipid second messenger, phosphatidylinositol 3, 4, 5-trisphosphate. J Biol Chem. 1998; 273:13375-8.
10. Stambolic V, Suzuki A, De La Pompa J L, Brothers G M, Mirtsos C, Sasaki T, et al. Negative regulation of PKB/Akt-dependent cell survival by the tumor suppressor PTEN. Cell 1998; 95:29-39.
11. Podsypanina K, Lee R T, Politis C, Hennessy I, Crane A, Puc J, et al. An inhibitor of mTOR reduces neoplasia and normalizes p70/S6 kinase activity in Pten+/− mice. Proc Natl Acad Sci USA 2001; 98:10320-5.
12. Mense S M, Barrows D, Hodakoski C, Steinbach N, Schoenfeld D, Su W, et al. PTEN inhibits PREX2-catalyzed activation of RAC1 to restrain tumor cell invasion. Sci Signal. 2015; 8: ra32-ra.
13. Wong J, Kim P, Peacock J, Yau T, Mui A-F, Chung S, et al. Pten (phosphatase and tensin homologue gene) haploinsufficiency promotes insulin hypersensitivity. Diabetologia 2007; 50:395-403.
14. Mendes-Pereira A M, Martin S A, Brough R, McCarthy A, Taylor J R, Kim J S, et al. Synthetic lethal targeting of PTEN mutant cells with PARP inhibitors. EMBO Mol Med. 2009; 1:315-22.
15. Shtivelman E, Sussman J, Stokoe D. A role for PI 3-kinase and PKB activity in the G2/M phase of the cell cycle. Curr Biol. 2002; 12:919-24.
16. Bassi C, Ho J, Srikumar T, Dowling R, Gorrini C, Miller S, et al. Nuclear PTEN controls DNA repair and sensitivity to genotoxic stress. Science 2013; 341:395-9.
17. Cory J G, Cory A H. Critical roles of glutamine as nitrogen donors in purine and pyrimidine nucleotide synthesis: asparaginase treatment in childhood acute lymphoblastic leukemia. In Vivo 2006; 20:587-9.
18. Stamato T D, Patterson D. Biochemical genetic analysis of pyrimidine biosynthesis in mammalian cells. II. Isolation and characterization of a mutant of Chinese hamster ovary cells with defective dihydroorotate dehydrogenase (E C 1.3. 3.1) activity. J Cell Physiol. 1979; 98:459-68.
19. Greene S, Watanabe K, Braatz-Trulson J, Lou L. Inhibition of dihydroorotate dehydrogenase by the immunosuppressive agent leflunomide. Biochem Pharmacol. 1995; 50:861-7.
20. Munier-Lehmann Hln, Vidalain P—O, Tangy Fdr, Janin Y L. On dihydroorotate dehydrogenases and their inhibitors and uses. J Med Chem. 2013; 56:3148-67.
21. Jiao J, Wang S, Qiao R, Vivanco I, Watson P A, Sawyers C L, et al. Murine cell lines derived from Pten null prostate cancer show the critical role of PTEN in hormone refractory prostate cancer development. Cancer Res. 2007; 67:6083-91.
22. Stratikopoulos E E, Dendy M, Szabolcs M, Khaykin A J, Lefebvre C, Zhou M-M, et al. Kinase and BET inhibitors together clamp inhibition of PI3K signaling and overcome resistance to therapy. Cancer Cell 2015; 27:837-51.
23. Stemke-Hale K, Gonzalez-Angulo A M, Lluch A, Neve R M, Kuo W-L, Davies M, et al. An integrative genomic and proteomic analysis of PIK3C A, PTEN, and AKT mutations in breast cancer. Cancer Res. 2008; 68:6084-91.
24. Puc J, Parsons R. PTEN loss inhibits CHK1 to cause double stranded-DNA breaks in cells. Cell Cycle 2005; 4:927-9.
25. Tibbetts R S, Cortez D, Brumbaugh K M, Scully R, Livingston D, Elledge S J, et al. Functional interactions between BRCA1 and the checkpoint kinase ATR during genotoxic stress. Genes Dev. 2000; 14:2989-3002.
26. Zou L, Elledge S J. Sensing DNA damage through ATRIP recognition of RPA-ssDNA complexes. Science 2003; 300:1542-8.
27. Kumagai A, Lee J, Yoo H Y, Dunphy W G. TopBP1 activates the ATR-ATRIP complex. Cell 2006; 124:943-55.
28. Liu K, Graves J D, Scott J D, Li R, Lin W-C. Akt switches TopBP1 function from checkpoint activation to transcriptional regulation through phosphoserine binding-mediated oligomerization. Mol Cell Biol. 2013; 33:4685-700.
29. Liu K, Paik J C, Wang B, Lin F T, Lin W C. Regulation of TopBP1 oligomerization by Akt/PKB for cell survival. EMBO J. 2006; 25:4795-807.
30. Toledo Luis I, Altmeyer M, Rask M-B, Lukas C, Larsen Dorthe H, Povlsen Lou K, et al. ATR prohibits replication catastrophe by preventing global exhaustion of RPA. Cell 2013; 155:1088-103.
31. Shechter D, Costanzo V, Gautier J. ATR and ATM regulate the timing of DNA replication origin firing. Nat Cell Biol. 2004; 6:648-55.
32. Keniry M, Parsons R. The role of PTEN signaling perturbations in cancer and in targeted therapy. Oncogene 2008; 27:5477-85.
33. White R M, Zon L I. DHODH regulates transcriptional elongation during melanoma evolution: insights from zebrafish screening. Cancer Res. 2011; 71: LB-335.
34. Carey L, Winer E, Viale G, Cameron D, Gianni L. Triple-negative breast cancer: disease entity or title of convenience? Nat Rev Clin Oncol. 2010; 7:683-92.
35. Bosch A, Eroles P, Zaragoza R, Vina J R, Lluch A. Triple-negative breast cancer: molecular features, pathogenesis, treatment and current lines of research. Cancer Treat Rev. 2010; 36:206-15.
36. Gierach G L, Burke A, Anderson W F. Epidemiology of triple negative breast cancers. Breast Dis. 2010; 32:5-24.
37. Saphner T, Tormey D C, Gray R. Annual hazard rates of recurrence for breast cancer after primary therapy. J Clin Oncol. 1996; 14:2738-46.
38. Hirshfield K M, Ganesan S. Triple-negative breast cancer: molecular subtypes and targeted therapy. Curr Opin Obstet Gynecol. 2014; 26:34-40.
39. Ma C X, Luo J, Ellis M J. Molecular profiling of triple negative breast cancer. Breast Dis. 2010; 32:73-84.
40. Gasparini P, Cascione L, Fassan M, et al. microRNA expression profiling identifies a four microRNA signature as a novel diagnostic and prognostic biomarker in triple negative breast cancers. Oncotarget 2014; 5:1174-84.
41. Bramati A, Girelli S, Torri V, et al. Efficacy of biological agents in metastatic triple-negative breast cancer. Cancer Treat Rev. 2014; 40:605-13.
42. Guan X, Ma F, Fan Y, Zhu W, Hong R, Xu B. Platinum-based chemotherapy in triple-negative breast cancer: a systematic review and meta-analysis of randomized-controlled trials. Anti-Cancer Drugs 2015; 26:894-901.
43. Mrklic I, Pogorelic Z, Capkun V, Tomic S. Expression of androgen receptors in triple negative breast carcinomas. Acta Histochem. 2013; 115:344-8.
44. Gucalp A, Tolaney S, Isakoff S J, et al. Phase II trial of bicalutamide in patients with androgen receptor-positive, estrogen receptor-negative metastatic breast cancer. Clin Cancer Res. 2013; 19:5505-12.
45. Pusztai L, Karn T, Safonov A, Abu-Khalaf M M, Bianchini G. New strategies in breast cancer: immunotherapy. Clin Cancer Res. 2016; 22:2105-10.
46. Teschner S, Burst V. Leflunomide: a drug with a potential beyond rheumatology. Immunotherapy 2010; 2:637-50.
47. van Riel P L, Smolen J S, Emery P, et al. Leflunomide: a manageable safety profile. J Rheumatol. 2004; 71:21-4.
48. Millikan R C, Newman B, Tse C K, et al. Epidemiology of basal-like breast cancer. Breast Cancer Res Treat. 2008; 109:123-39.
49. Mall J W, Myers J A, Xu X, Saclarides T J, Philipp A W, Pollmann C. Leflunomide reduces the angiogenesis score and tumor growth of subcutaneously implanted colon carcinoma cells in the mouse model. Chirurg 2002; 73:716-20.
50. Astin J W, Jamieson S M, Eng T C, et al. An in vivo antilymphatic screen in zebrafish identifies novel inhibitors of mammalian lymphangiogenesis and lymphatic-mediated metastasis. Mol Cancer Ther. 2014; 13:2450-62.
51. Hail N, Jr., Chen P, Kepa J J, Bushman L R. Evidence supporting a role for dihydroorotate dehydrogenase, bioenergetics, and p53 in selective teriflunomide-induced apoptosis in transformed versus normal human keratinocytes. Apoptosis 2012; 17:258-68.
52. Baumann P, Mandl-Weber S, Volkl A, et al. Dihydroorotate dehydrogenase inhibitor A771726 (leflunomide) induces apoptosis and diminishes proliferation of multiple myeloma cells. Mol Cancer Ther. 2009; 8:366-75.
53. Zhu S, Yan X, Xiang Z, Ding H F, Cui H. Leflunomide reduces proliferation and induces apoptosis in neuroblastoma cells in vitro and in vivo. PLOS ONE 2013; 8:e71555.
54. Alhefdhi A, Burke J F, Redlich A, Kunnimalaiyaan M, Chen H. Leflunomide suppresses growth in human medullary thyroid cancer cells. J Surg Res. 2013; 185:212-6.
55. Dietrich S, Kramer O H, Hahn E, et al. Leflunomide induces apoptosis in fludarabine-resistant and clinically refractory CLL cells. Clin Cancer Res. 2012; 18:417-31.
56. Hail N, Jr., Chen P, Bushman L R. Teriflunomide (leflunomide) promotes cytostatic, antioxidant, and apoptotic effects in transformed prostate epithelial cells: evidence supporting a role for teriflunomide in prostate cancer chemoprevention. Neoplasia 2010; 12:464-75.
57. O'Donnell E F, Kopparapu P R, Koch D C, et al. The aryl hydrocarbon receptor mediates leflunomide-induced growth inhibition of melanoma cells. PLOS ONE 2012; 7:e40926.
58. Jin U H, Lee S O, Safe S. Aryl hydrocarbon receptor (AHR)-active pharmaceuticals are selective AHR modulators in MDA-MB-468 and BT474 breast cancer cells. J Pharmacol Exp Ther. 2012; 343:333-41.
59. Puc J, Parsons R. PTEN loss inhibits CHK1 to cause double stranded-DNA breaks in cells. Cell Cycle 2005; 4:927-9.
60. Eisenhauer E A, Therasse P, Bogaerts J, et al. New response evaluation criteria in solid tumours: revised RECIST guideline (version 1.1). Eur J Cancer 2009; 45:228-47.
61. Saal L H, Johansson P, Holm K, et al. Poor prognosis in carcinoma is associated with a gene expression signature of aberrant PTEN tumor suppressor pathway activity. Proc Natl Acad Sci USA 2007; 104:7564-9.
62. Erickson J M, Adelstein K E, Letzkus L C. A feasibility study to measure physical activity, fatigue, sleep-wake disturbances, and depression in young adults during chemotherapy. J Adolesc Young Adult Oncol. 2014; 3:37-41.
63. Cohen S, Cannon G W, Schiff M, et al. Two-year, blinded, randomized, controlled trial of treatment of active rheumatoid arthritis with leflunomide compared with methotrexate. Utilization of leflunomide in the treatment of rheumatoid arthritis trial investigator group. Arthritis Rheumatol. 2001; 44:1984-92.
64. Strand V, Cohen S, Schiff M, et al. Treatment of active rheumatoid arthritis with leflunomide compared with placebo and methotrexate. Leflunomide rheumatoid arthritis investigators group. Arch Intern Med. 1999; 159:2542-50.
65. Smolen J S. Efficacy and safety of the new DMARD leflunomide: comparison to placebo and sulfasalazine in active rheumatoid arthritis. Scand J Rheumatol. 1999; 112:15-21.
66. Osiri M, Shea B, Robinson V, et al. Leflunomide for treating rheumatoid arthritis. Cochrane Database Syst Rev. 2003: CD002047.
67. Yuan M, Breitkopf S B, Yang X, et al. A positive/negative ion-switching, targeted mass spectrometry-based metabolomics platform for bodily fluids, cells, and fresh and fixed tissue. Nat Protoc. 2012; 7:872-81.
68. Kamburov A, Cavill R, Ebbels™, et al. Integrated pathway-level analysis of transcriptomics and metabolomics data with IMPaLA. Bioinformatics. 2011; 27:2917-8.
69. Subramanian A, Tamayo P, Mootha V K, et al. Gene set enrichment analysis: A knowledge-based approach for interpreting genome-wide expression profiles. Proc Natl Acad Sci USA 2005; 102:15545-50.
70. Mootha V K, Lindgren C M, Eriksson K-F, et al. PGC-la-responsive genes involved in oxidative phosphorylation are coordinately downregulated in human diabetes. Nat Genet. 2003; 34:267-73.
71. Rozman B. Clinical pharmacokinetics of leflunomide. Clin Pharmacokinet. 2002; 41:421-30.
72. Breedveld F, Dayer J. Leflunomide: mode of action in the treatment of rheumatoid arthritis. Ann Rheum Dis. 2000; 59:841-9.

1. A method of treating a cancer in a human subject in need thereof, the method comprising:
(a) obtaining a cell of the cancer from the human subject;
(b) testing the obtained cancer cell for the presence of wild-type or mutant phosphate and tensin homolog (PTEN);
(c) determining that the cancer cell is partially deficient for PTEN or active PTEN relative to a wild-type cell of the same species and tissue type, or that the cancer cell does not express detectable PTEN or active PTEN; and
(d) administering to the human subject a dihydroorotate dehydrogenase (DHODH) inhibitor,
wherein the DHODH inhibitor is leflunomide, brequinar, redoxal, S-2678, or teriflunomide; and
wherein the cancer is selected from the group consisting of breast cancer, a glioblastoma, prostate cancer, uterine cancer, ovarian cancer, pancreatic cancer, melanoma, renal cell carcinoma, bladder cancer, colorectal cancer, lymphoma, leukemia, and oropharyngeal cancer.

2. The method of claim 1, wherein the cancer is partially deficient for PTEN relative to a wild-type tissue of the same species and tissue type.

3. (canceled)

4. The method of claim 1, wherein step (c) comprises determining that the cancer cell does not comprise detectable PTEN.

5. (canceled)

6. The method for claim 1, wherein step (c) comprises determining that the cancer cell comprises a PTEN mutation in the germline or primary tumor.

7. (canceled)

8. The method of claim 1, wherein the DHODH inhibitor is administered orally, parenterally, intradermally, subcutaneously, topically, or rectally.

9. The method of claim 1, further comprising treating the subject with one or more additional therapeutic regimens.

10. The method of claim 9, wherein the one or more additional therapeutic regimens are selected from the group consisting of surgery, chemotherapy, radiation therapy, hormone therapy, and immunotherapy.

11. (canceled)

12. The method of claim 1, wherein the PTEN mutant cancer developed as a result of an alteration of PTEN which occurred somatically during tumor initiation or progression or in the germline.

13. (canceled)

14. The method of claim 1, wherein the PTEN mutant cancer is a relapsed cancer or was refractory to one or more previous treatments.

15. (canceled)

16. A method for predicting the efficacy of a DHODH inhibitor in inducing DNA damage in a cancer in a human subject, the method comprising:
(a) testing a cell of the cancer obtained from the human subject for the presence of wild-type or mutant PTEN,
(b) predicting that a DHODH inhibitor would likely induce DNA damage in the cancer if the cell is partially deficient for PTEN or active PTEN relative to a wild-type cell of the same species and tissue type, or if the cell does not comprise detectable PTEN or active PTEN; and
(c) if the cancer cell is found to be partially deficient for PTEN or active PTEN relative to a wild-type cell of the same species and tissue type, or if the cancer cell does not express detectable PTEN or active PTEN, administering to the human subject the DHODH inhibitor;
wherein the DHODH inhibitor is leflunomide, brequinar, redoxal, S-2678, or teriflunomide; and
wherein the cancer is selected from the group consisting of breast cancer, a glioblastoma, prostate cancer, uterine cancer, ovarian cancer, pancreatic cancer, melanoma, renal cell carcinoma, bladder cancer, colorectal cancer, lymphoma, leukemia, and oropharyngeal cancer.

17. A method for predicting the efficacy of a DHODH inhibitor in treating a cancer in a human subject, the method comprising:
(a) testing a cell of the cancer obtained from a human subject for the presence of wild-type or mutant PTEN,
(b) predicting that a DHODH inhibitor would likely induce DNA damage in the cancer and thereby treat the cancer if the cell is partially deficient for PTEN or active PTEN relative to a wild-type cell of the same species and tissue type, or if the cell does not comprise detectable PTEN or active PTEN; and
(c) if the cancer cell is found to be partially deficient for PTEN or active PTEN relative to a wild-type cell of the same species and tissue type, or if the cancer cell does not express detectable PTEN or active PTEN, administering to the human subject the DHODH inhibitor;
wherein the DHODH inhibitor is leflunomide, brequinar, redoxal, S-2678, or teriflunomide; and
wherein the cancer is selected from the group consisting of breast cancer, a glioblastoma, prostate cancer, uterine cancer, ovarian cancer, pancreatic cancer, melanoma, renal cell carcinoma, bladder cancer, colorectal cancer, lymphoma, leukemia, and oropharyngeal cancer.

18.-22. (canceled)

23. The method of claim 16, wherein the cancer is breast cancer, wherein the breast cancer is triple-negative breast cancer.

24. The method of claim 17, cancer is breast cancer, and wherein the breast cancer is triple-negative breast cancer.

25. The method claim 16, wherein the cancer is a relapsed cancer or was refractory to one or more previous treatments.

26. The method of claim 17, wherein the cancer is a relapsed cancer or was refractory to one or more previous treatments.

27.-41. (canceled)
